(19) United States
(12) Patent Application Publication
(10) Pub. No.: US 2022/0401660 A1
(43) Pub. Date: Dec. 22, 2022

(54) PAIN-REDUCING INSERTION APPARATUS AND USES THEREOF

(71) Applicant: Joseph Choate BURKETT, Addison, TX (US)

(72) Inventor: Joseph Choate BURKETT, Addison, TX (US)

(21) Appl. No.: 17/761,124

(22) PCT Filed: Sep. 18, 2020
(86) PCT No.: PCT/US2020/051667

§ 371 Date: Mar. 16, 2022

Related U.S. Application Data
(60) Provisional application No. 62/902,894, filed on Sep. 19, 2019.

(51) Int. Cl. A61M 5/42; A61M 5/44

(52) U.S. Cl. CPC A61M 5/422; A61M 5/44; A61M 2230/50; A61M 2205/3368; A61M 2205/3606; A61M 2005/1585

(57) ABSTRACT

Provided are systems and methods for reducing pain during an injection or an insertion of an object into the skin of a subject.

### CROSS-REFERENCE

This application claims the benefit of U.S. Provisional Application No. 62/902,894, filed Sep. 19, 2019, which application is incorporated herein by reference.

The subject matter of this application is related to that of International Patent Application No. PCT/US2018/022506, filed Mar. 14, 2018; and U.S. Provisional Patent Application No. 62/471,168, filed Mar. 14, 2017, the contents of which are incorporated herein by reference in their entirety.

### BACKGROUND

Many people have aversion to injections due to their fear of needles. Such fear can have major negative consequences because the patients may refuse necessary treatments due to the anticipation of the pain. It would be particularly dire for people with chronic illnesses such as those who suffer from diabetes to avoid proper treatment due to their fear. Hence, it would be extremely beneficial to develop an insertion system that can effectively reduce the pain the patients suffer from during routine operations.

### SUMMARY

Provided herein are embodiments of an apparatus for reducing pain during puncturing of skin of a subject, the apparatus comprising: a cooling system comprising one or more cooling surfaces to contact the skin of the subject to cause a reduction in a temperature of the skin proximal to a puncture site; and a vibration system to provide vibrations to the skin proximal to the puncture site, wherein the vibrations and the reduction of the temperature of the skin are carried out simultaneously to reduce pain at the puncture site during puncturing of the skin.

In some embodiments, the reduction of temperature caused by the one or more cooling surfaces is approximately 10 to 20 degrees Celsius. In some embodiments, the reduction of temperature caused by the one or more cooling surfaces is approximately 10 degrees Celsius. In some embodiments, the one or more cooling surfaces cool the skin proximal to the puncture site to about 10 to 20 degrees Celsius. In some embodiments, the one or more cooling surfaces cool the skin proximal to the puncture site to about 15 to 20 degrees Celsius. In some embodiments, the one or more cooling surfaces cool the skin proximal to the puncture site to about 20 degrees Celsius. In some embodiments, the one or more cooling surfaces do not cool the skin proximal to the puncture site to less than 10 degrees Celsius. In some embodiments, the one or more cooling surfaces do not cool the skin proximal to the puncture site to less than 15 degrees Celsius. In some embodiments, the one or more cooling surfaces do not cool the skin proximal to the puncture site to less than 20 degrees Celsius. In some embodiments, the one or more cooling surfaces do not cool the skin proximal to the puncture site to less than 25 degrees Celsius.

In some embodiments, the temperature of the skin proximal to a puncture site is reduced as the one or more cooling surface are cooled to a target temperature. In some embodiments, the target temperature is approximately 0 to 10 degrees Celsius. In some embodiments, the one or more cooling surfaces are cooled to a first temperature prior to being cooled to the target temperature. In some embodiments, the first temperature is less than the target temperature. In some embodiments, the skin is punctured as the one or more cooling surfaces being cooled to the target temperature. In some embodiments, the one or more cooling surfaces begin cooling after being contacted to the skin.

In some embodiments, the apparatus comprises a mechanism to determine if the one or more surfaces are in contact with the skin. In some embodiments, the mechanism to determine if the one or more surfaces are in contact with the skin comprises a pressure sensor. In some embodiments, the pressure sensor is a piezoelectric pressure sensor.

In some embodiments, the apparatus further comprises one or more temperature sensors to measure the temperature of the skin proximal to the puncture site. In some embodiments, the apparatus further comprises one or more temperature sensors to measure the temperature of the one or more cooling surfaces. In some embodiments, the vibrations have a frequency of approximately 100 Hertz to 1000 Hertz. In some embodiments, the vibrations have a frequency of approximately 400 Hertz to 600 Hertz. In some embodiments, the vibrations have an amplitude of 0.1 G to 100 G. In some embodiments, the vibrations have an amplitude of 0.1 G to 100 G.

In some embodiments, the apparatus comprises an aperture surrounded by the one or more cooling surfaces, wherein the aperture is placed at the puncture site and wherein an object which punctures the skin is placed through the aperture and into the puncture site. In some embodiments, the aperture comprises a radius of about 0.5 millimeters to 4 millimeters. In some embodiments, the aperture comprises a radius of about 0.5 millimeters to 1 millimeter.

In some embodiments, the aperture is fully surrounded by the one or more cooling. In some embodiments, the aperture is partially surrounded by the one or more cooling surfaces, such that the puncture site is also partially surrounded by the one or more cooling surfaces.

In some embodiments, the apparatus comprises a handle and an applicator provided at one end of the handle, wherein the applicator comprises the one or more cooling surfaces. In some embodiments, the applicator comprises an aperture surrounded by the one or more cooling surfaces, wherein the aperture is placed at the puncture site and wherein an object which punctures the skin is placed through the aperture and into the puncture site.

In some embodiments, the apparatus further comprises a heat sink in thermal communication with the one or more cooling surfaces. In some embodiments, operation of the cooling system is reversible, such that heat stored in the heat sink is utilized to warm the one or more cooling surfaces. In some embodiments, the one or more cooling surfaces are warmed in preparation for a subsequent puncturing of the skin. In some embodiments, the subsequent puncturing of the skin occurs at a subsequent puncture site a different location from the puncture site. In some embodiments, the aperture comprises a radius of about 0.5 millimeters to 4 millimeters. In some embodiments, the aperture comprises a radius of about 0.5 millimeters to 1 millimeter. In some embodiments, the aperture is fully surrounded by the one or more cooling surfaces, such that the puncture site is also fully surrounded by the one or more cooling surfaces.

In some embodiments, the aperture is partially surrounded by the one or more cooling surfaces, such that the puncture site is also partially surrounded by the one or more cooling surfaces. In some embodiments, the applicator is contoured proximal to the aperture to help guide the object which punctures the skin to the puncture site. In some embodiments, the object which punctures the skin is a needle. In some embodiments, the needle is a hypodermic needle.

Provided herein are embodiments of a method of reducing pain a puncture site located on a skin of a subject, the method comprising: applying a contact surface to the skin, proximal to the puncture site; cooling the contact surface; vibrating the contact surface; and puncturing the skin at the puncture site as the contact surface is vibrating and cooling.

In some embodiments, the steps of cooling the contact surface and vibrating the contact surface are conducted after the contact surface is applied to the skin. In some embodiments, the puncturing the skin is conducted as the contact surface is being cooled. In some embodiments, the step of cooling the contact surface comprises cooling the contact surface to a target temperature. In some embodiments, the target temperature is approximately 0 to 10 degrees Celsius. In some embodiments, the cooling surface is cooled to a first temperature prior to being cooled to the target temperature. In some embodiments, the first temperature is less than the target temperature.

In some embodiments, the method further comprises a step of monitoring the temperature of the skin surface. In some embodiments, monitoring the temperature of the skin surface is carried out by a temperature sensor. In some embodiments, cooling of the contact surface is stopped if the temperature of the skin surface is determined to be less than 20 degrees Celsius. In some embodiments, cooling of the contact surface is stopped if the temperature of the skin surface is determined to be less than 15 degrees Celsius. In some embodiments, cooling of the contact surface is stopped if the temperature of the skin surface is determined to be less than 10 degrees Celsius.

In some embodiments, the method further comprises a step of monitoring the temperature of the contact surface. In some embodiments, monitoring the temperature of the contact surface is carried out by a temperature sensor. In some embodiments, cooling of the contact surface is stopped if the temperature of the skin surface is determined to be less than 10 degrees Celsius. In some embodiments, cooling of the contact surface is stopped if the temperature of the skin surface is determined to be less than 5 degrees Celsius. In some embodiments, cooling of the contact surface is stopped if the temperature of the skin surface is determined to be less than 0 degrees Celsius.

In some embodiments, the method further comprises a step of warming the cooling surface after the step of puncturing the skin in preparation for a subsequent puncturing of the skin. In some embodiments, the subsequent puncturing of the skin is conducted at a subsequent puncture site a location on the skin different from a location of the puncture site.

Provided herein are embodiments of a pain reducing insertion apparatus, comprising: a needle holder configured to move the needle in an axial direction with respect to a body of the insertion apparatus to insert the needle in an insertion site of an individual during deployment of the needle; and a pain reducing system comprising a cooling system and a vibration system, wherein the cooling system is configured to cool a contact surface by conduction and the vibrator is configured to cause the contact surface to vibrate, wherein the contact surface is configured to be in a close proximity to the insertion site during the deployment of the needle.

In some embodiments, the apparatus further comprises a catheter configured to couple with the needle during the deployment of the needle and wherein the catheter is inserted in the insertion site at end of the deployment. In some embodiments, the catheter is configured to be coupled to a drug delivery device. In some embodiments, the drug delivery device is an infusion set. In some embodiments, the pain reducing system does not move with respect to the body of the insertion apparatus during the deployment of the needle. In some embodiments, the cooling system is an active cooling system. In some embodiments, the active cooling system comprises a thermoelectric cooling system. In some embodiments, the cooling system is a passive cooling system. In some embodiments, the cooling system is configured to maintain the contact surface at a constant temperature.

In some embodiments, the cooling system is configured to maintain the contact surface at fluctuating temperatures. In some embodiments, the fluctuating temperatures is pre-programmed.

In some embodiments, the vibrating system is configured to vibrate the contact surface at a constant frequency. In some embodiments, the vibrating system is configured to vibrate the contact surface at fluctuating frequencies. In some embodiments, the fluctuating frequencies is pre-programmed.

Provided herein are embodiments of a method of reducing pain during insertion of a needle at an insertion site on a skin of a patient, the method comprising: contacting a contact surface to the skin proximal to the insertion site; vibrating the contact surface with a vibration system; cooling the contact surface with a cooling system; and inserting a needle into the insertion site while the contact surface is cooling and vibrating.

In some embodiments, the needle is coupled to a catheter, and the method further comprises a step of inserting the catheter after the needle is inserted into the insertion site. In some embodiments, the steps of cooling the contact surface and vibrating the contact surface are conducted after the contact surface is applied to the skin. In some embodiments, the step of cooling the contact surface comprises cooling the contact surface to a target temperature. In some embodiments, the target temperature is approximately 0 to 10 degrees Celsius.

In some embodiments, the cooling surface is cooled to a first temperature prior to being cooled to the target temperature. In some embodiments, the first temperature is less than the target temperature.

In some embodiments, the method further comprises a step of monitoring the temperature of the skin proximal to the insertion site. In some embodiments, monitoring the temperature of the skin is carried out by a temperature sensor. In some embodiments, cooling of the contact surface is stopped if the temperature of the skin is determined to be less than 20 degrees Celsius. In some embodiments, cooling of the contact surface is stopped if the temperature of the skin is determined to be less than 15 degrees Celsius. In some embodiments, cooling of the contact surface is stopped if the temperature of the skin is determined to be less than 10 degrees Celsius.

In some embodiments, the method further comprises a step of monitoring the temperature of the contact surface. In some embodiments, monitoring the temperature of the contact surface is carried out by a temperature sensor. In some embodiments, cooling of the contact surface is stopped if the temperature of the skin surface is determined to be less than 10 degrees Celsius. In some embodiments, cooling of the contact surface is stopped if the temperature of the skin surface is determined to be less than 5 degrees Celsius. In some embodiments, cooling of the contact surface is stopped if the temperature of the skin surface is determined to be less than 0 degrees Celsius.

Provided herein are embodiments of a system for reducing pain during puncturing of skin of a subject, the system comprising: a cooling unit comprising: a cooling system comprising one or more cooling plates provided at a distal end of the cooling unit, and a vibration system comprising a vibrational motor provided at the distal end of the cooling unit; and an applicator removably attached to the distal end of the cooling unit, the end cap comprising: a cooling surface in thermal communication with the one or more cooling plate of the cooling unit, wherein the cooling system of the cooling unit cools the cooling surface of the applicator.

In some embodiments, the cooling system further comprises a heat sink to dissipate heat. In some embodiments, further comprising a thermal mass in thermal communication with the heat sink, such that heat generated by the cooling system is dissipated into the thermal mass through the heat sink. In some embodiments, the system further comprises a handle which is removably attached to a proximal end of the cooling unit, wherein the handle comprises the thermal mass.

In some embodiments, the cooling unit further comprises a microprocessor, and wherein the handle further comprises one or more buttons to receive an input for activating the cooling system, the vibration system, or both. In some embodiments, the contact comprises a chip, wherein the chip transmits information to the microprocessor of the cooling unit to specify one or more pain reduction parameters. In some embodiments, the pain reduction parameters comprise a vibration frequency, a vibration amplitude, a vibration mode, a target temperature of the cooling surface, and combinations thereof.

In some embodiments, the system further comprises an end cap, wherein the end cap is removably attached to a proximal end of the handle, and wherein the end cap comprises a fan to dissipate heat from the thermal mass. In some embodiments, the system further comprises a protective sleeve to envelope the cooling unit. In some embodiments, the system is reversible, and wherein the thermal mass is cooled prior to the attachment of the handle to the cooling unit to cool the one or more cooling plates of the cooling unit. In some embodiments, the cooling unit further comprises a rechargeable battery, and wherein the thermal mass recharges the rechargeable battery.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts a perspective view of an insertion device comprising a needle cartridge, according to one embodiment.

FIG. 2 depicts a side view of an embodiment of an insertion device body.

FIG. 3 depicts a cross-sectional view of an embodiment of an insertion device body taken along lines 12-12.

FIG. 4 depicts a cross-sectional view of the device body shown in FIG. 3 taken along lines 14-14.

FIG. 5 depicts a top perspective view of an embodiment of an insertion cartridge.

FIG. 6 depicts an exploded view of an embodiment of an insertion cartridge.

FIG. 7 depicts a cross-sectional view of an embodiment of an insertion cartridge.

FIG. 8 depicts a top perspective view of an embodiment of an assembled needle shuttle tube during the needle deployment.

FIG. 9 depicts a cross-sectional view of an embodiment of a needle shuttle tube without the other related components.

FIG. 10 depicts a top perspective cross-sectional view of an embodiment of a needle shuttle tube without the other related components.

FIG. 11 depicts a top perspective view of an embodiment of a bottom portion of the needle shuttle tube without the other related components.

FIG. 12 depicts a cross-sectional view of an embodiment of a bottom portion of the needle shuttle tube without the other related components.

FIG. 13 depicts a top perspective view of an embodiment of a needle holder and a needle.

FIG. 14 depicts a cross-sectional view of an embodiment of a needle holder and needle.

FIG. 15 depicts a top perspective view of an embodiment of an adhesive cap and a catheter.

FIG. 16 depicts a cross-sectional view of an embodiment of an assembled insertion device comprising a device body and an insertion cartridge, before the needle insertion.

FIG. 17 depicts a cross-sectional view of another embodiment of an assembled insertion device comprising a device body and insertion cartridge, before needle and catheter insertion.

FIG. 18 depicts a cross-sectional view of an embodiment of an insertion cartridge during needle and catheter insertion.

FIG. 19 depicts a cross-sectional view of an embodiment of an insertion cartridge, after the adhesive cap is released following the insertion of catheter and the withdrawal of the needle.

FIG. 20 depicts a cross-sectional view of an embodiment of an assembled insertion device comprising the device body and insertion cartridge during the needle and the catheter insertion.

FIG. 21 depicts a cross-sectional view of an embodiment of an assembled insertion device comprising the device body and the insertion cartridge, after the adhesive cap is released following the insertion of catheter and the withdrawal of the needle.

FIG. 22 depicts the temperature response skin area with a corresponding cooling curve from a cooling surface applied to the skin, according to some embodiments.

FIG. 23A depicts a pain reduction apparatus, according to some embodiments.

FIG. 23B depicts a pain reduction apparatus, according to some embodiments.

FIG. 24A depicts a pain reduction system, according to some embodiments.

FIG. 24B depicts a pain reduction system, according to some embodiments.

FIG. 25 depicts a cooling unit of a pain reduction system, according to some embodiments.

FIG. 26A depicts a handle component of a pain reduction system, according to some embodiments.

FIG. 26B depicts a handle component of a pain reduction system, according to some embodiments.

FIG. 27 depicts an end cap component of a pain reduction system, according to some embodiments.

### DETAILED DESCRIPTION

Provided herein, in some embodiments, are methods and systems for reducing perceived pain associated with puncturing a skin surface. Puncturing of the skin surface may be necessary for administering an injection of a medicament. The systems and methods described herein may be utilized in to reduce perceived pain during various procedures, such as insertion of needles for acupuncture, Botox injections, hair transplantation, hormone injections, insulin injections, vaccinations, IV starts, administering a medicament, inserting a subcutaneous apparatus, and the like. The systems and methods herein may also be used to reduce pain during a jet injections such as lidocaine introduced through the skin via jet injection. or other transdermal injections.

### I. Pain Reduction During Attachment of a Medical Device

Disclosed herein, in certain embodiments, is a pain reducing insertion device for facilitating the insertion of or attachment of a medical device beneath the surface of the skin or to the surface of the skin while reducing the perceived pain associated with the insertion or the attachment. Devices disclosed herein may improve the performance and safety of medical device attachment and may reduce the associated perceived pain.

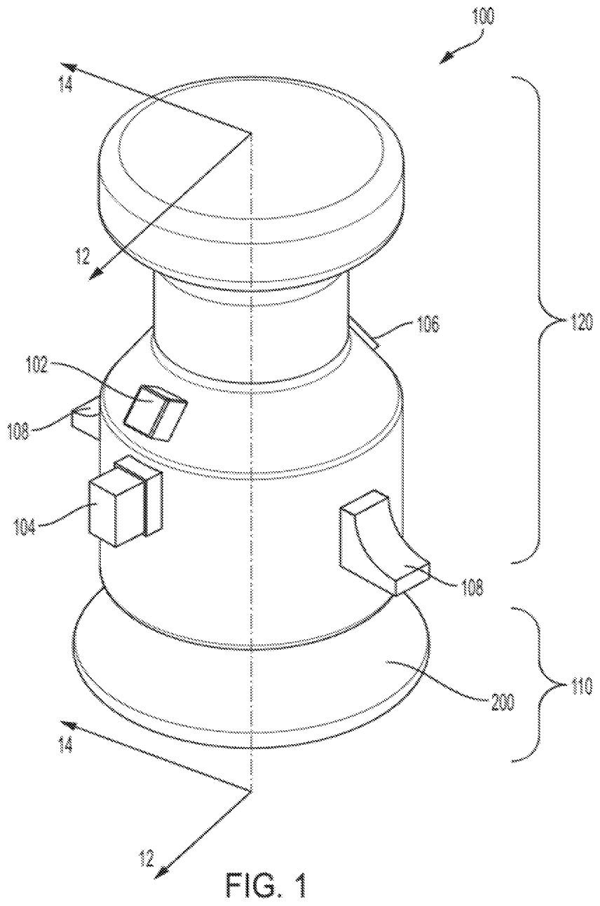

FIG. 1 illustrates one example of an embodiment of the insertion device 100, which includes a device body 120 and an insertion cartridge 110. The insertion device 100 can be used to insert or attach a medical device to a patient, while reducing the perceived pain related to the insertion. In some embodiments, the insertion cartridge contains an insulin needle set, but various other needle cartridges could insert or attach various other medical devices or catheters. The medical device can be syringe or infusion set. The infusion set can comprise one or more pumps. In some embodiment, the infusion set coupled to the insertion device can be used to administer insulin.

The medical devices or catheters inserted or attached by the device 100 may comprise the components used to deliver medications, fluids, or other substances. The medical devices or catheters inserted or attached could alternatively be used to remove, sample, sense, or examine bodily fluids, liquids, or any other substances, or other components or molecules thereof. The medical device or catheter inserted or attached can comprise components, including but not limited to, onboard electronic components, transcutaneous sensors, other sensors, a display screen, indicator lights, or other indicators. In some embodiments, the components can have timing functions, information processing capabilities, ability communicate to other devices, ability to attach other electronic components, and/or ability to attach other non-electronic components. In certain embodiments, the medical device or attached/inserted catheter may comprise components related to medication delivery such as medication storage components, medication delivery components, pumps, attachments for pumps, tubing, attachments for tubing, or other medication related attachments. The medical device or catheter inserted or attached may comprise one or more of the following components: components for removal, sampling, sensing, and examining bodily fluids, liquids, or other substances, and other components or molecules thereof, components for delivering medications, fluids, or other substances, components related to medication delivery related such as medication storage components, medication delivery components, pumps, attachments for pumps, tubing, attachments for tubing, and other medication related attachments. Some nonlimiting examples of these medical devices can include insulin sets as stated above, as well as continuous glucose monitors, combination devices that both sense glucose levels and deliver insulin, and various other intermittent or continuously monitoring devices and medication delivery devices.

FIG. 1 illustrates an embodiment of the insertion device 100 that comprise a device body 120 and an insertion cartridge 110. The exterior aspect of the device body can include a housing of the device body 120, activation button for the pain reduction mechanisms such as 102, an activation button for medical device insertion 104, and a trigger to release the insertion cartridge 108 from the body of the device.

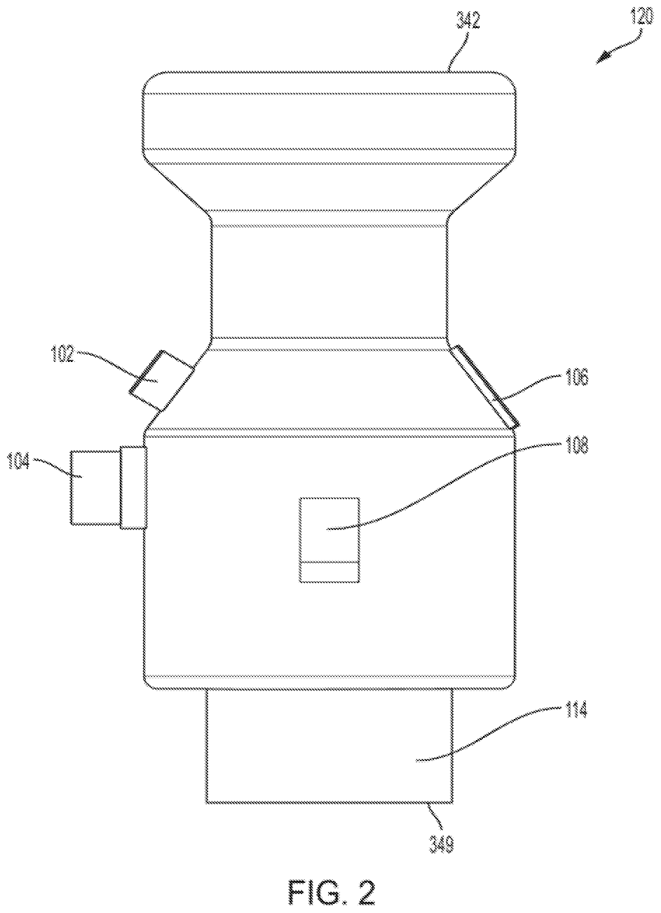

FIG. 2 Illustrates the insertion device 100 without an attached insertion cartridge 120. In some embodiments, the upper aspect of the device body 342 can be gripped by the user and can be faced away from the surface of the skin, and the lower aspect of the device body 344 can be directed towards the skin surface of the patient. The outer wall of the lower shaft 114 can be seen in FIG. 2. In some embodiments, when the device cartridge 110 is attached to the device body 120 as in FIG. 1, the outer wall of the lower shaft may not be seen externally and can lie within the cylindrical shaft of the insertion cartridge 154, adjacent to the lateral wall of the cylindrical shaft 156.

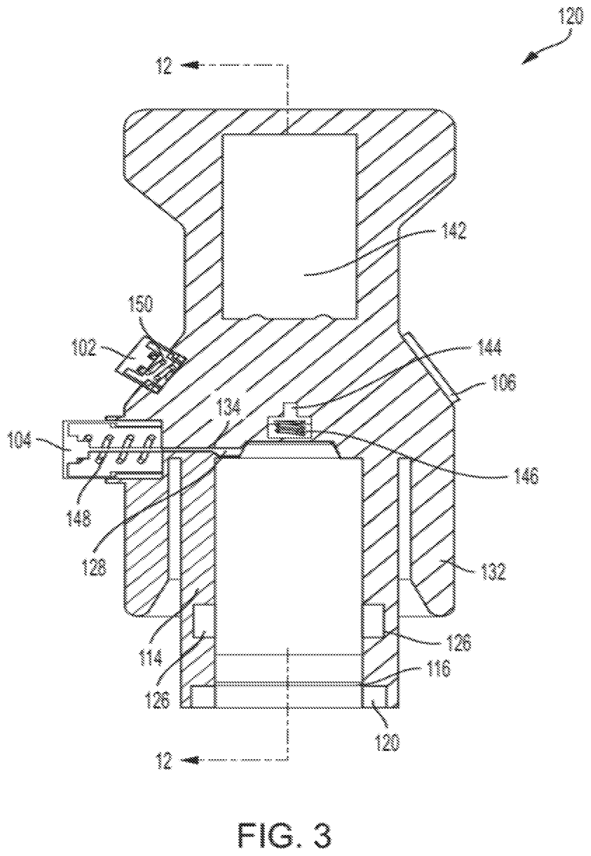

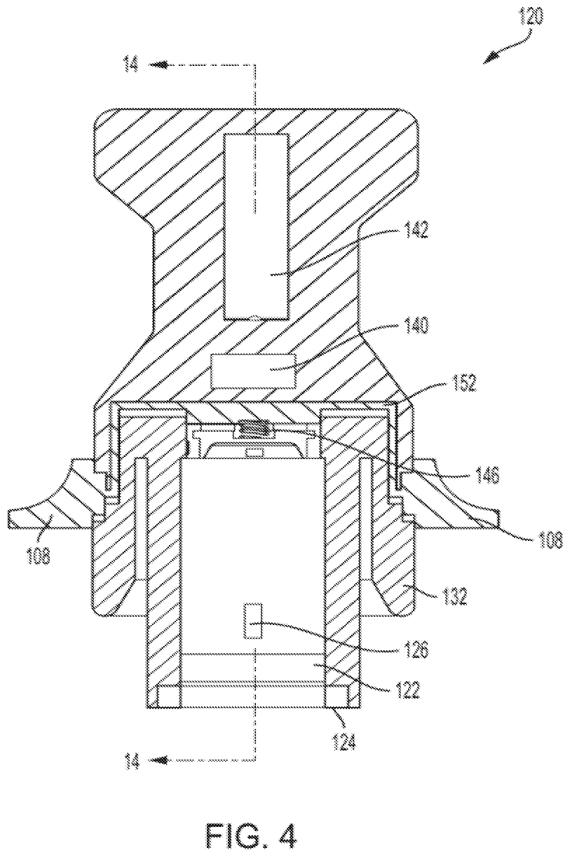

Referring to FIGS. 3 and 4, these images illustrate cross-sectional side views of the device body 120 cut across the plane formed by line 12 in FIG. 3, and by line 14 in FIG. 4. In FIGS. 3 and 4, the lower shaft of the device body 114 is shown, as well as the thermoelectric cooling plate 120 on the lower medial portion. The thermoelectric connection 116 can bridge the cooling side of the thermoelectric cooler 120 and hot side of the thermoelectric cooler. In a certain embodiment, device further comprises a vibration component 126. The upper aspects of the lower cylinder wall 114 can be used as a heat sink or heat dissipater so that the cooling plate can absorb more heat at lower voltages.

In some embodiments, a separate heat sink component may be provided. In some embodiments, a docking station is provided having one or more heat sinks to contact the one or more cooling surfaces for rapid heat dissipation. In some embodiments, after use, the cooling system runs in reverse to dissipate heat through the cooling surfaces. This may also help warm the cooling surfaces in preparation for reducing pain for subsequent insertions.

Also illustrated in FIG. 3 are the cartridge stabilizing wings 132 that can provide additional support for the insertion cartridge 110. The stabilizing wings 132 can have asymmetry such that a specific orientation of the insertion cartridge is required to load it. The activation button for medical device insertion 104 is shown in FIG. 3 as well. When not depressed, the spring opposing button for needle insertion activation 148 can keep an opposing pressure on the activation button 104 while at the same time keeping the needle insertion activation spud/key 128 in a retracted position. The activation spud/key can sit within the shaft for medical device insertion activation 134. When the activation button for medical device insertion is pressed by the user while an insertion cartridge 110 (not shown in FIG. 3) is attached to the device body 120, the insertion cartridge can be activated and the enclosed medical device can be compelled towards the skin.

A biometric scanner 106 is illustrated in FIG. 3. The biometric scanner 106 illustrated in this embodiment can use digital imaging to scan a patient's wrist band barcode, the signals of which are sent via wires (not shown), to the onboard computer 140 (shown in FIG. 4), which can then determine if the appropriate wrist band correlating to a specific patient to which the device has been assigned to has been scanned. If the computer 140 determines that the wrist band does correspond to the correct patient, an electromechanically lock (not show in these illustrations) can be released. The biometric scanner could use one or more of the following types of scanner to image and sense different types of data, including but not limited to, a fingerprint scanner, a retinal scanner, facial recognition, among various others. In some embodiments, the device can use a form of password protection, via a display screen, mechanical mechanism, or remote activation. The lock preventing the use on a non-designated person or patient can take various forms as well. It can be electro-mechanical as described in this embodiment, it can be magnetic, it can prevent the attachment of the device to the insertion cartridge, it can block the transmission of electronic signals, and/or it can block mechanically block the insertion activation, among various types of other mechanisms and configurations.

Also illustrated in FIGS. 3 and 4 are a cross-section of the aperture for cartridge ejection 144, the spring opposing the cartridge ejection mechanism 146, and the cartridge ejection lever 152. The cartridge ejection lever 152 can be attached to cartridge ejection trigger 108. After the insertion cartridge has been activated and completed its use, the cartridge can be ejected by the user, by pressing downward on the cartridge ejection trigger 108. In certain embodiments, other mechanisms by which to eject the used cartridge can be used, such as a mechanism that automatically release the cartridge after use, electronic signal to a mechanical or magnetic mechanism, various other mechanical mechanisms, pneumatic, electronic, or other types or mechanisms. In other embodiments, the insertion cartridge and the body of the device are permanently attached and both discarded after one each.

Another aspect of this embodiment as illustrated in FIGS. 3 and 4 can include the rechargeable battery pack 142. In some embodiments, the device body could have an access point for recharging this battery pack (not shown in the images), a wireless recharger, charging stand, some other type of connection to an AC or DC current, solar energy capture, and mechanical to electrical energy capture, among various other variations.

In some embodiments, the insertion device can use nonelectronic mechanisms in one or all of the mechanisms in which it performs. In some embodiments the vibration could be created by use of a mechanical device, such as a trigger that can cause a mass to spin which thereby causes vibration, pneumatic powered vibration, as well as various other types of non-electronic vibration mechanisms. In some embodiments, the lower surface of the device can change shape in uniform or non-uniform fashion, such that it stimulates nerves and decreases perceived pain.

In some embodiments, the thermal electric cooler can be replaced with a thermal mass. The thermal mass can comprise one or more of the following substances: one or more types of metal, plastic, rubber, frozen liquid, cold but not frozen liquid, carbon fiber, natural or synthetic substances, or other materials, and may have layers or combinations of multiple materials. The layers of material between the thermal mass surface and/or thermoelectric cooler surface and tissue surface one or more of the following substances: one or more types of metal, plastic, rubber, frozen liquid, cold but not frozen liquid, carbon fiber, natural or synthetic substances, or other materials, and may have layers or combinations of multiple materials. If the thermal mass does not have an ideal cooling curve, such as ice which causes the outermost surface of human tissue to cool too rapidly such that it causes pain and discomfort when applied to the skin even when separated from the skin by some materials such as plastic, one or more materials can be added between the skin and the thermal mass that modifies the cooling curve, thereby changing the rate of change in temperature and the total amount of change caused by absorption of heat from tissue to a more effective cooling curve, which can make for more effective pain reduction system and less likely to damage or kill the tissue cells. The thermal mass can be cooled to a set temperature before being used, such as placing it in a standard freezer, a standard refrigerator, or it can be cooled by a device specific cooling device, so that the temperature of the cold mass may not so cold as to be unsafe to human (or animal) tissue and cells, and can have a certain heat transfer rate, so that it can cool the tissue surface in an effective pain reducing and predictable fashion.

The use of thermoelectric cooling devices along with the concurrent use of a vibration device can be significantly more effective at decreasing the perceived pain associated transdermal needle insertion and device attachment than either of those two mechanisms individually. It has been shown that it can be fairly difficult to safely get tissue at the depth at which pain receptors lie within the skin (100 μM below the skin surface) cooled to the temperatures at which peripheral pain receptors are inhibited, as the surface temperature must be significantly colder than that to achieve the inhibition temperature of the pain receptors. But by using the two mechanisms in combination which allows for synergy, it might not be required to reach the significantly colder temps to achieve a more effective pain reduction. Gate controlled mechanisms of pain control might contribute to the synergy of these mechanisms, but it is possible another physiological process causes the synergy. One embodiment of the thermoelectric cooler disclosed herein can use a power of 10-20 W and vibration of approximately 200-500 Hz. These ranges can be extended.

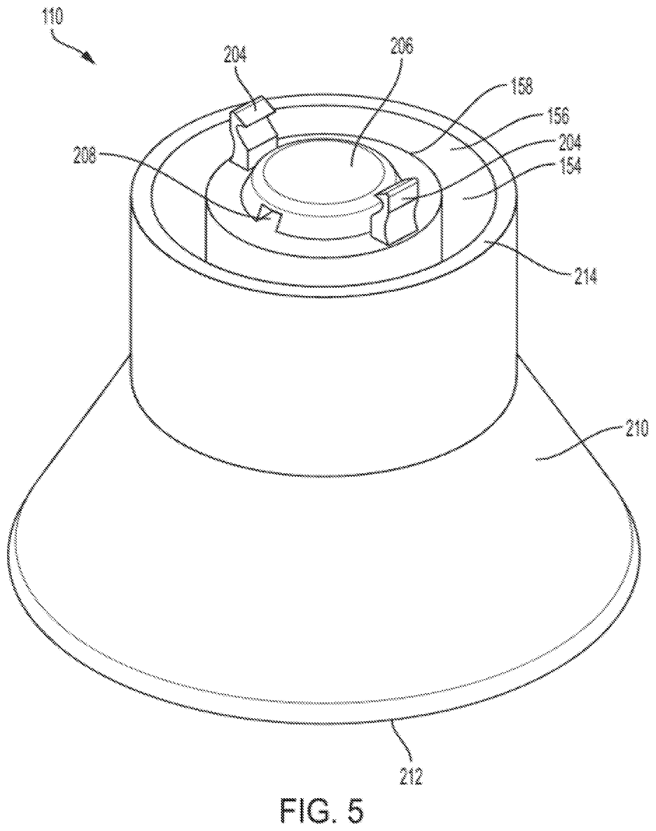

FIG. 5 illustrates one embodiment of the insertion cartridge 110 while not attached to the device body 120. In this embodiment, the insertion cartridge can comprise the components used to insert the needle and catheter of an insulin set, as well as withdraw and lock the needle in a safe position. In another embodiment, one or more of the mechanisms can be completed by the device body.

In this embodiment of the insertion device, device body attachment hooks 204 are located on the top surface of the insertion cartridge 110, and can reversibly attach the insertion cartridge 110 to the device body 120. The hooks 204 can be snapped into place by matching the top features of the insertion cartridge and aligning them with the bottom features of the device body 120, then slidably insert the top portion of the insertion cartridge into the bottom surface of the device body 120 until the hooks snap into place. In other embodiments, the attachment hooks can be part of the device body 110, and the attachment of the insertion cartridge can be accomplished using various other mechanisms such as twisting or screwing the cartridge into the device body 110.

FIG. 5 shows the cylindrical shaft of the insertion cartridge 154, the lateral wall of the cylindrical shaft of the insertion cartridge 156, and the medial of the cylindrical shaft of the insertion cartridge 158. The shape of these aspects can match the shape and size of the lower cylinder of the device body 114. In an alternative embodiment, the shape of these components may be non-cylindrical, such that even on insertion of the insertion cartridge, at the inferior most aspect of the cylindrical shaft 154, is the pain reduction conduction area 224.

Also illustrated in FIG. 5 is the lateral wedge portion 210 of the insertion cartridge 110. In this embodiment, the lateral wedge portion 210 can make the insertion cartridge easier for the user to hold and manipulate the insertion cartridge, it increases the surface area of the bottom of the cartridge which also can make the assembled device 100 more stable and easier to hold and handle, and can contain the tubing and attachment for the insulin pump to the pump set (though this portion is not directly illustrated in this figure. In other embodiments, this wedged shape portion 210 of the insertion cartridge can contain one or more portion of medical devices that can be inserted or attached, or may not be present at all.

In certain embodiments, a portion of the insertion cartridge 110 may also include one or more of the following items attached to the outside or contained within: zero, one, or multiple thermal mass, thermoelectric cooler, vibration component, shape changing component, oscillating component, electronic stimulation component, chemically anesthetizing component, aerosolizing component, tactile stimulation component, suction component, and various other pain reducing components.

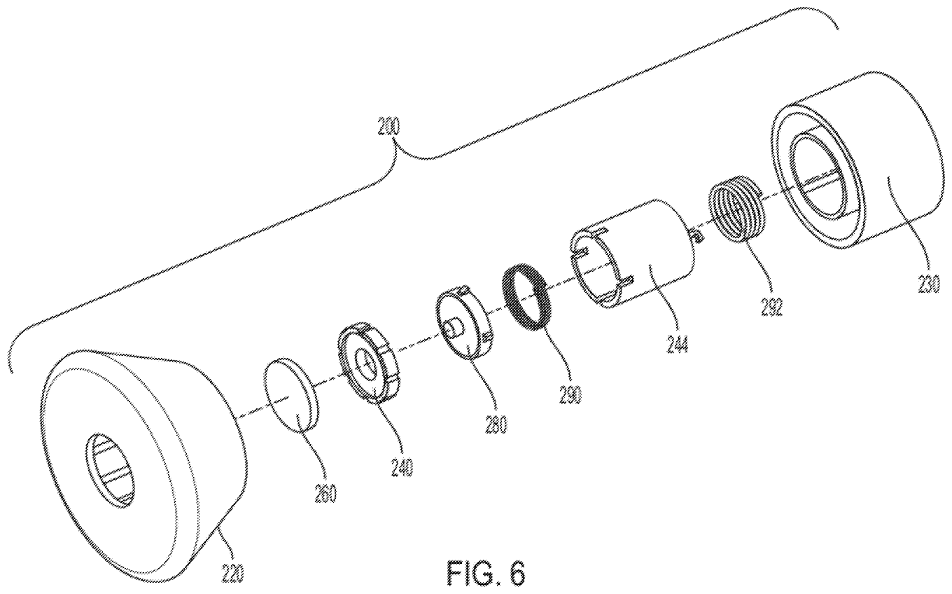

FIG. 6. Illustrates an exploded view of the components of the insertion cartridge 110. In some embodiments, the exploded view include the upper portion of the insertion cartridge body 230, the spring for downward drive of the needle shuttle tube 292, the upper portion of the needle shuttle tube 244, needle holder return spring 290, the needle holder and needle 280, the lower portion of the needle shuttle tube 240, the adhesive patch and transcutaneous catheter of the insulin set 260, and the lower portion of the insertion cartridge body 220. In certain embodiments, one or some of these components can be components of the device body and not the insertion cartridge.

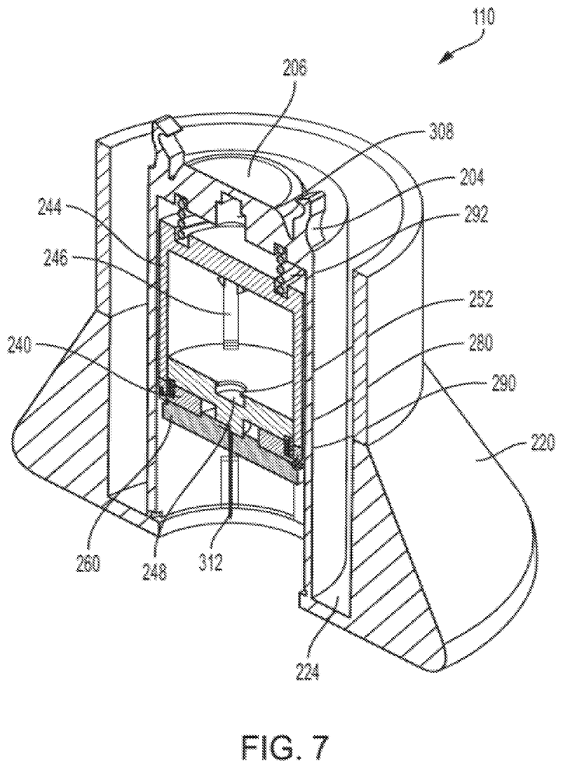

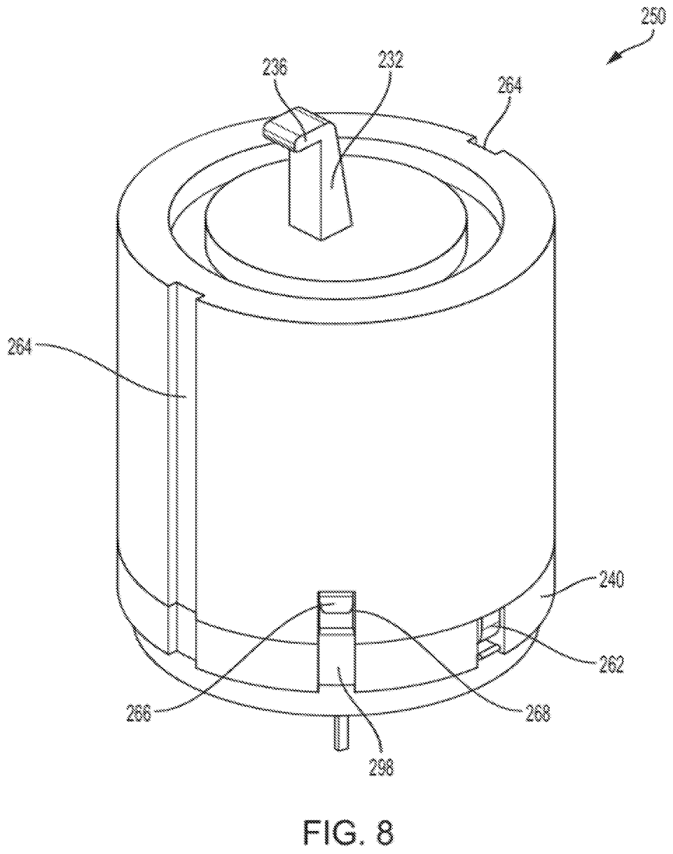

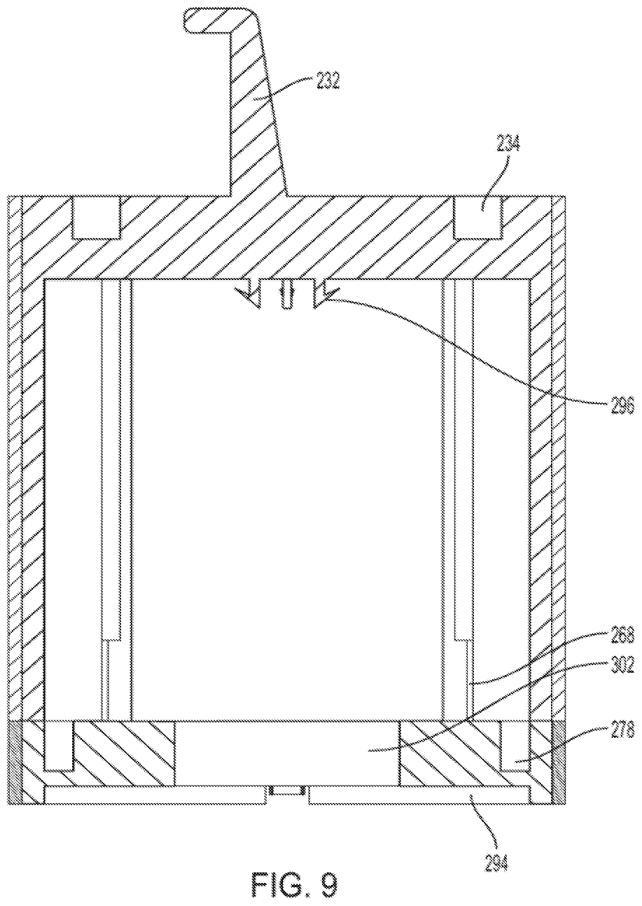

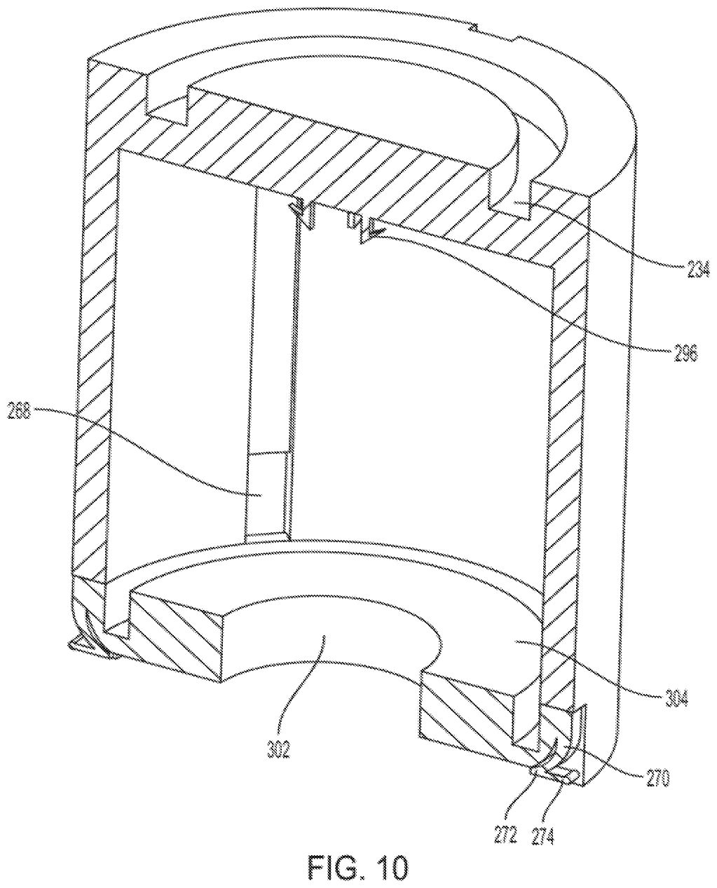

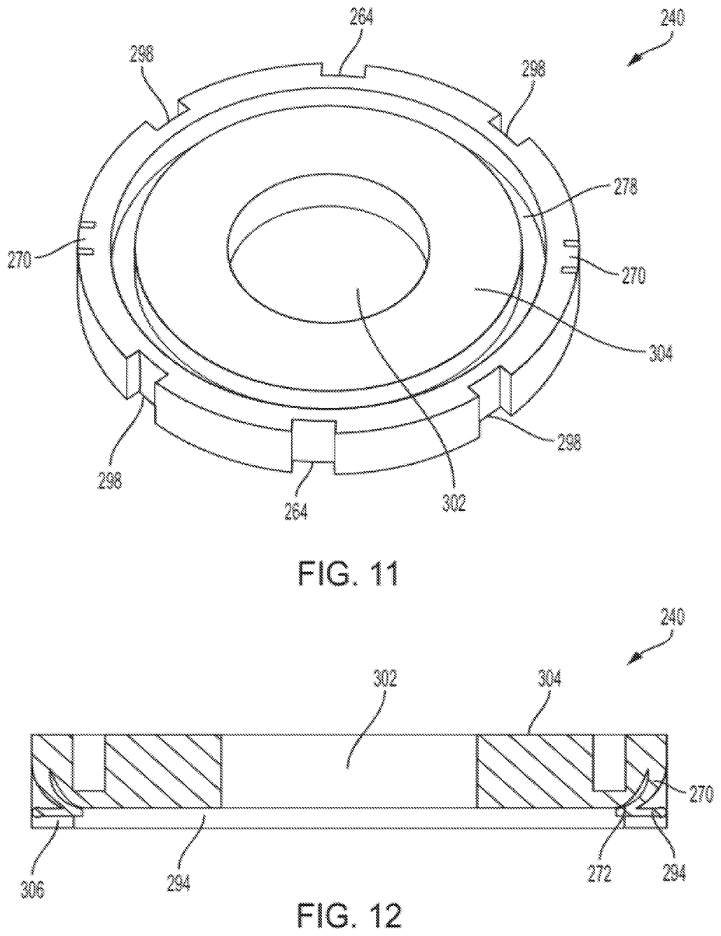

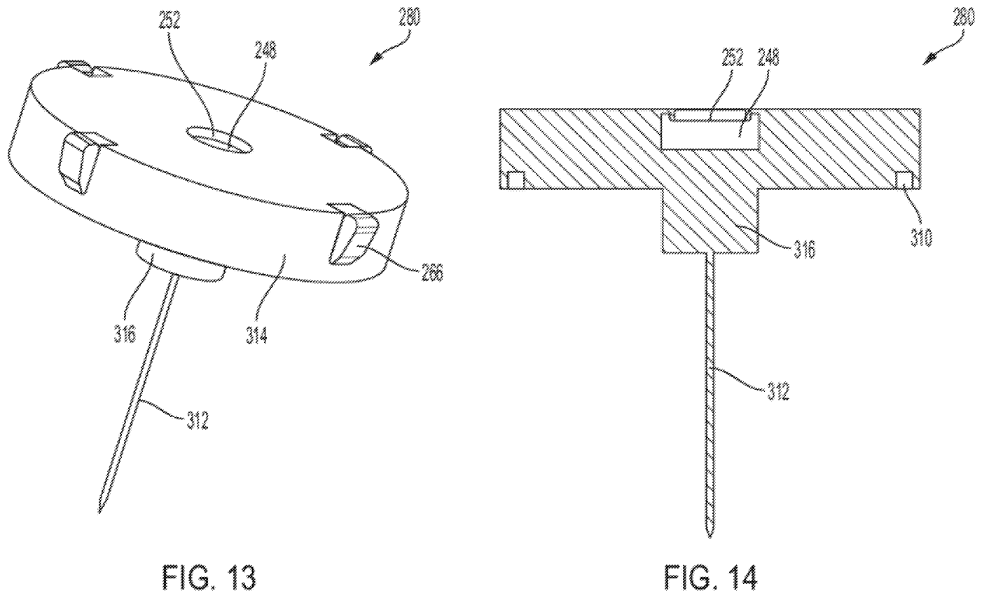

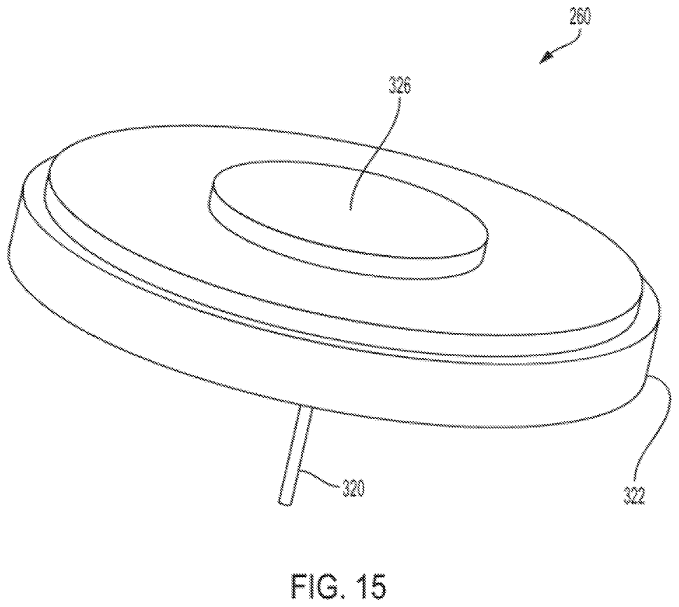

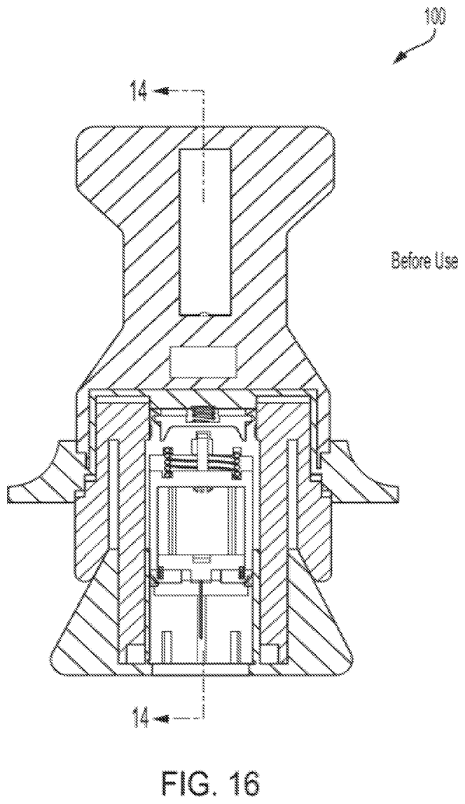

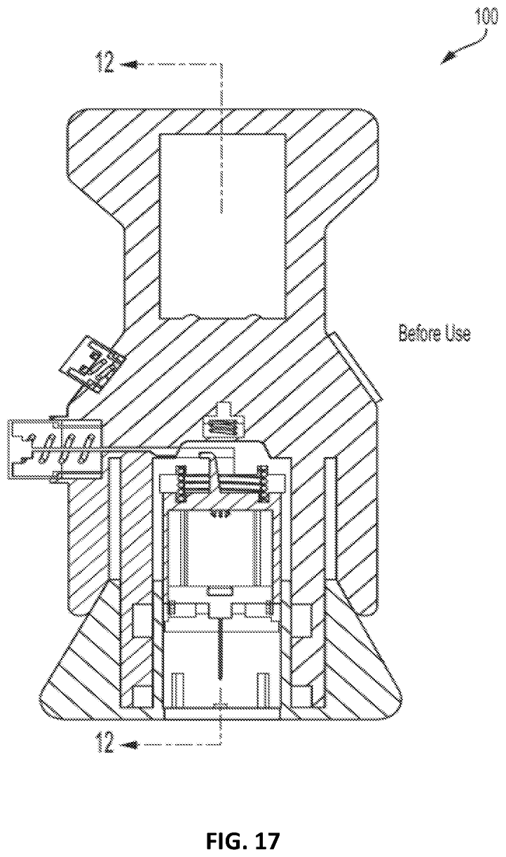

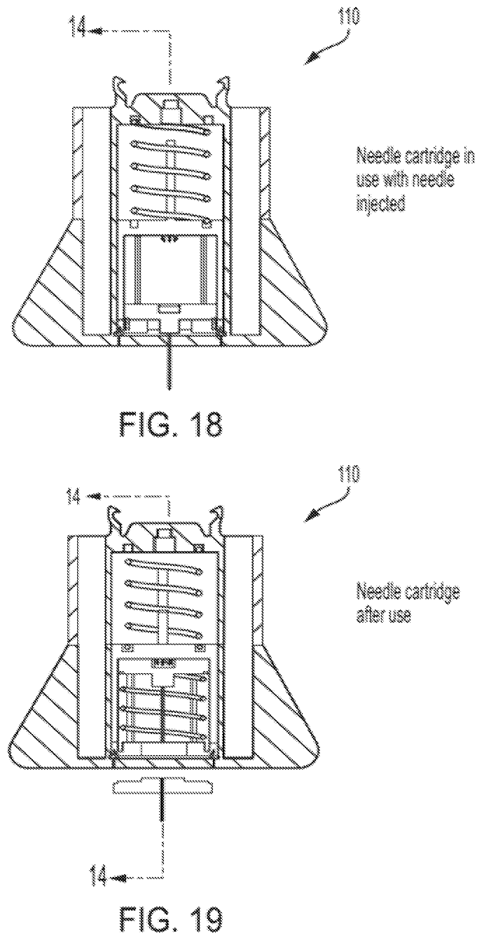

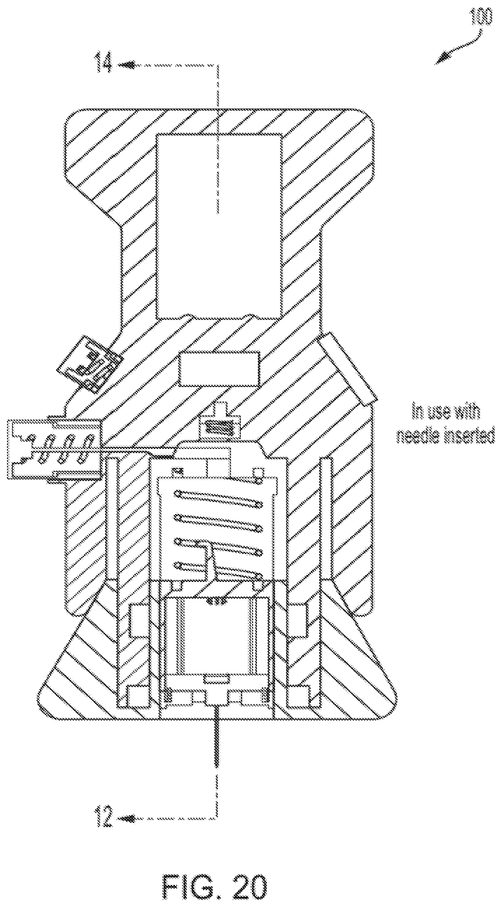

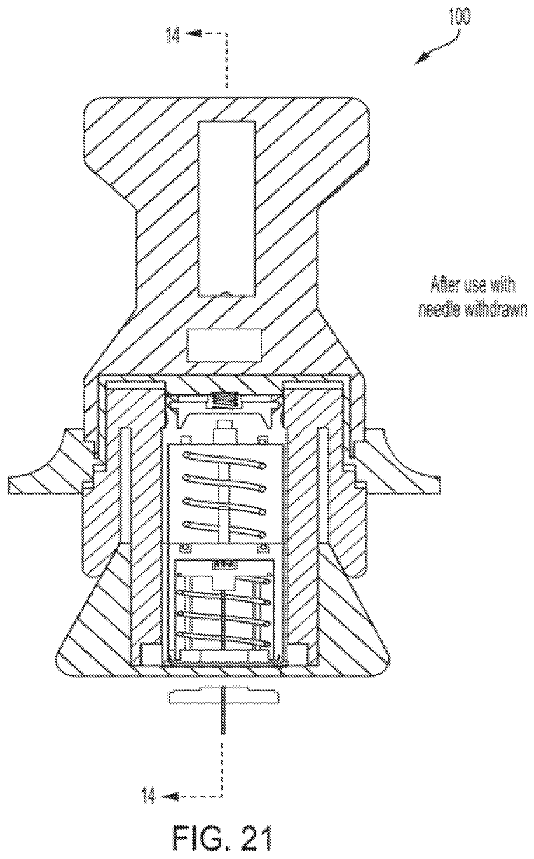

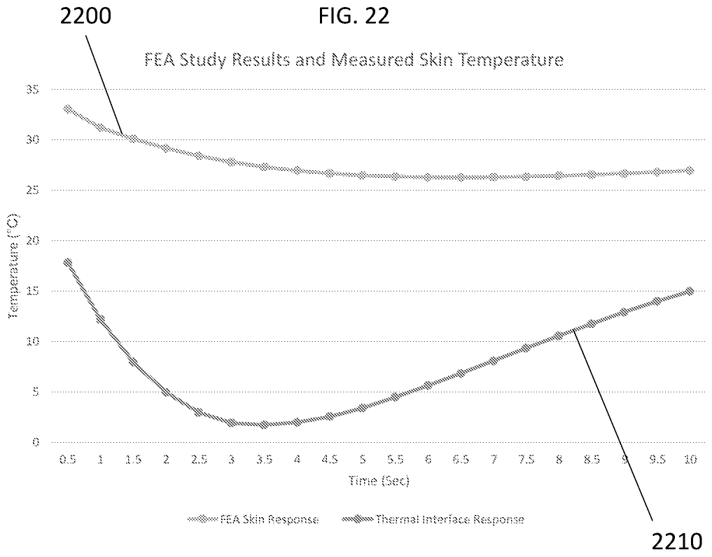

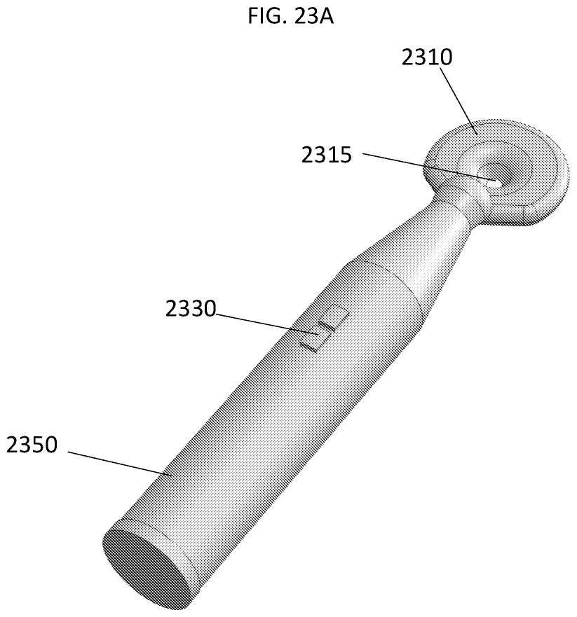

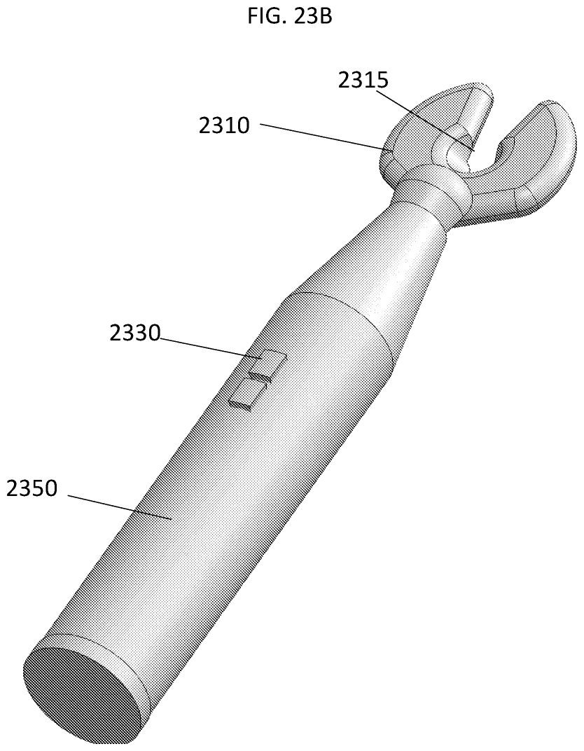

FIG. 7 illustrates a perspective cross-sectional view of the insertion cartridge of this embodiment. As in FIG. 6, the device body attachment hooks 204 can be present. The device can comprise the needle insertion activation guard 206 as well. At the center of the needle insertion activation arm is the internal recess for needle activation prevention arm 308. It is within this recess that the needle shuttle tube upper anchor arm 232 can reside before use of the cartridge, and can be seen in FIG. 8.

Also illustrated in FIG. 7. is the spring driving the needle shuttle tube downward 292, the needle holder slideback guide 246, the needle holder lock cavity 248, the needle holder lock wing 252, the needle holder return spring 290, and the pain reduction conduction area 224, the upper portion of the needle shuttle tube 244, the needle holder and needle, the lower portion of the needle shuttle tube 240.

FIG. 8 demonstrates the needle shuttle tube and its contents 250, separated from the insertion cartridge body. The device can comprise the needle shuttle tube upper anchor arm 232 which can be used to prevent the insertion cartridge from activating prematurely. When the needle shuttle tube and its contents are present within the body of the insertion cartridge, and before use, the upper anchor arm 232 can be firmly hooked onto the upper surface of the insertion cartridge within the aperture for insertion activation 208 and internal recess for needle activation prevention arm 308, and can oppose the downward forces related to the downward drive spring 292. When the device insertion activation button 104 is pressed by the user, the needle insertion activation spud/key 128 which can be attached on its lateral end to the activation button, can be advanced medially though the shaft for medical device insertion activation shaft 134, and into the aperture for insertion activation 208, to the point at which the spud/key flexes the needle shuttle tube arm medially and the foot of the needle shuttle tube anchor 236 breaks free medially from the top surface of the insertion cartridge and the needle shuttle tube and its contents 250, can be driven downward towards the tissue surface. [0048] Also illustrated in FIG. 8, the lateral guide grooves 264 can guide the needle guide tube 250 straight down, when the downward drive spring 292 propels the needle shuttle tube 250 downward towards the surface of the patient's skin. As the needle guide tube 250 nears the bottom of the central shaft 282, the groove for dislodging shoulder for needle holder in 298 can slide over the surface of the needle holder arms dislodging shoulder 284 just after which time the dislodging shoulders slide into the needle holder arms aperture 268 and into direct contact with the needle holder arms 266, which can then be displaced inward and upward. This action can free the needle holder and needle from its bottom most location. At the same time as the needle holder arms are being dislodged, the needle 312 and catheter 320 can reach their maximum depth under the surface of the skin, and the adhesive cap 322 can be at the same time coming into contact with the skin. As the needle shuttle tube reaches its maximum downward distance, it can be stopped by the lower stopping wedge 286. The adhesive cap grip arms 270 located on opposing lateral positions of the lower portion of the needle shuttle tube can have a spring type force tending them outward towards the lateral walls of the center cylinder 282, but the adhesive cap arms 270 and feet 274 can be held in a medial position until the needle shuttle tube 250 reaches its lowest position on insertion, at which time the arms 270 and feet 274 can be pushed to a more lateral position, as they enter the lower cap release recess 216. As the adhesive cap grip arms 270 flex laterally into the recess, the movement of the adhesive cap grip arms 270 and feet 274 can cause a lateral and downward motion of the adhesive cap grip shoulders 272 thereby causing a downward motion of the adhesive cap 322 and catheter 320, pressing it harder onto the skin while at the same time releasing it from the grip of the adhesive cap grip shoulders 272 and the its position within the lower portion of the needle shuttle tube. With the needle holder arms 266 free of the needle holder arm aperture 268, the needle holder and needle can be driven upward by the needle holder needle holder return spring 290, until they too are locked into an upward position and the upper needle holder lock hooks 296 snap under the needle holder lock wing 252. This can lock the needle and needle holder in an upward position, preventing needle sticks while removing and disposing of the insertion cartridge.

In some embodiments, a pain reduction device is configured to work in conjunction with an existing device. In some embodiments, the pain reduction device is configured to attach to an existing device. In some embodiments, the existing device is a disposable device. An example of an existing device may be a surgical staple gun, wherein the device comprises some component which is punctures the skin where the pain reduction during operation may be useful.

### II. Active Cooling Curve

In some embodiments, a cooling system is configured to cool a surface which is placed on an area of the skin to receive a puncture, insertion, or injection. In some embodiments the cooling system comprises a Peltier cooler. The surface which is cooled by the cooling system and contacts the skin may be referred to as the contact surface or cooling surfaces. In some embodiments, a system comprises one or more contact or cooling surfaces. In some embodiments, one or more cooling surfaces of a pain reduction device are configured to cool a skin surface for perceived pain reduction. In some embodiments, the cool surfaces are configured to activate A-delta nociceptors. A-delta nociceptors are considered fast acting myelinated pain receptors in the skin which may override other pain receptors. The A-delta nociceptors which we are aiming to trigger may be found in the skin at a depth between 50-100 micrometers (um). In some embodiments, the depth at which the A-delta nociceptors are located may depend. on the person and area of the body. Activation of the A-delta nociceptors by cooling has been experimentally verified to suppress pain when the A-delta reduced to 20° C.

Normally, human skin rests at a temperature of approximately 30° C. Reducing that temperature by only 10° C. to 20° C. may be sufficient to reduce perceived pain. It has been found that cooling the skin to 20° C. at the 50-100 um depth reduces pain threshold and detection by about 50% and reports of pain by 75%. Therefore, it may be possible to configure one or more cooling surfaces to reduce perceived pain while staying above temperature thresholds which may damage the skin or cause pain. For example, a skin temperature of approximately 14-23° C. may cause a painful sensation to be perceived from cooling of the skin. The specific temperature at which pain is perceived may vary based on the patient or the location at which the skin is cooled. Further a skin temperature of 7° C. may cause numbness, a temperature of −0.6° C. may cause freezing, potentially damaging the skin surface, and temperatures of −4.8° C. or less may cause frostbite, which will damage the skin surface. In some embodiments, the active cooling surface requires about 10 to 25 watts to per square inch to provide the required temperature change. In some embodiments, the power is supplied at 3 to 9 volts. The wattage and voltage may be adjusted to increase or decrease the time to reach a target temperature, first temperature, second temperature, of a contact surface or skin surface, as disclosed herein.

Therefore, the cooling surface of the systems and devices disclosed herein may be configured or regulated such that the cooling surface does not damage the skin or cause pain at the cooled skin surface. In some embodiments, one or more cooling surfaces are controlled such that the skin surface is cooled to about 5° C. to about 30° C. In some embodiments, one or more cooling surfaces are controlled such that the skin surface is cooled to about 30° C. to about 27° C., about 30° C. to about 25° C., about 30° C. to about 23° C., about 30° C. to about 20° C., about 30° C. to about 18° C., about 30° C. to about 15° C., about 30° C. to about 12° C., about 30° C. to about 10° C., about 30° C. to about 6° C., about 30° C. to about 5° C., about 27° C. to about 25° C., about 27° C. to about 23° C., about 27° C. to about 20° C., about 27° C. to about 18° C., about 27° C. to about 15° C., about 27° C. to about 12° C., about 27° C. to about 10° C., about 27° C. to about 6° C., about 27° C. to about 5° C., about 25° C. to about 23° C., about 25° C. to about 20° C., about 25° C. to about 18° C., about 25° C. to about 15° C., about 25° C. to about 12° C., about 25° C. to about 10° C., about 25° C. to about 6° C., about 25° C. to about 5° C., about 23° C. to about 20° C., about 23° C. to about 18° C., about 23° C. to about 15° C., about 23° C. to about 12° C., about 23° C. to about 10° C., about 23° C. to about 6° C., about 23° C. to about 5° C., about 20° C. to about 18° C., about 20° C. to about 15° C., about 20° C. to about 12° C., about 20° C. to about 10° C., about 20° C. to about 6° C., about 20° C. to about 5° C., about 18° C. to about 15° C., about 18° C. to about 12° C., about 18° C. to about 10° C., about 18° C. to about 6° C., about 18° C. to about 5° C., about 15° C. to about 12° C., about 15° C. to about 10° C., about 15° C. to about 6° C., about 15° C. to about 5° C., about 12° C. to about 10° C., about 12° C. to about 6° C., about 12° C. to about 5° C., about 10° C. to about 6° C., about 10° C. to about 5° C., or about 6° C. to about 5° C. In some embodiments, one or more cooling surfaces are controlled such that the skin surface is cooled to about 30° C., about 27° C., about 25° C., about 23° C., about 20° C., about 18° C., about 15° C., about 12° C., about 10° C., about 6° C., or about 5° C. In some embodiments, one or more cooling surfaces are controlled such that the skin surface is cooled to at least about 30° C., about 27° C., about 25° C., about 23° C., about 20° C., about 18° C., about 15° C., about 12° C., about 10° C., or about 6° C. In some embodiments, one or more cooling surfaces are controlled such that the skin surface is cooled to at most about 27° C., about 25° C., about 23° C., about 20° C., about 18° C., about 15° C., about 12° C., about 10° C., about 6° C., or about 5° C.

In some embodiments, use of higher temperatures during the cooling of skin surface to reduce pain not only reduces the chances of damaging the skin, but also reduces the energy required to cool the cooling surface which contacts the skin, thus providing a more efficient system which is just as effective as a system which cools a cooling surface to a lower temperature. Use of higher temperatures may also allow for less battery drain during use. In some embodiments, the use of higher temperatures allows the system or apparatus to be used more often. In some embodiments, the use of higher temperatures allows the system or apparatus to be used more per battery or per battery charge. This may be especially useful for repetitive needle insertion procedures such as Botox injections or acupuncture.

In some embodiments, contact of the one or more cooling surfaces with the skin as a temperature of the cooling surface or surfaces are lowered increases the reduction of perceived pain. In some embodiments, reduction of the cooling surface temperature does not begin until the cooling surface is brought in contact with the skin. Cooling of the active cooling surfaces may be initiated manual, e.g. by a user pressing a button, or automatically. In some embodiments, cooling of one or more active cooling surface is automated as a mechanism detects that the one or more cooling surfaces have been placed on a skin. The mechanism may include one or more pressure sensors, such as a piezoelectric pressure sensor, to detect when a cooling surface is in contact with the skin. In some embodiments, placing a cooling surface placed against the skin may cause a contact located an opposite surface to abut an electrical contact, thereby triggering an electrical connection to activate cooling of a cooling surface. A spring may be provided between the contacts, such that the electrical circuit is closed when the cooling surface is removed from the skin. In some embodiments, an electrical sensor detects a change in resistance as a cooling surface contacts the skin, which then initiates cooling of an active cooling surface.

A decrease in the level of perceived pain may correlate with the total change in temperature of the skin or a cooling surface applied to the skin. A larger change in temperature of the skin or a cooling surface applied to the skin may correlate to a greater decrease in pain experienced by a patient or subject during the insertion of a needle or other device to puncture the skin. However, the total change in temperature may be limited to prevent pain from the temperature of the contact surface or damage to the skin. In some embodiments, the cooling surface is not cooled prior to contacting the skin. In some embodiments, the cooling surface is warmed prior to contacting the skin. In some embodiments, the contact or cooling surface is warmed to about room temperature (i.e. about 23° C.). In some embodiments, the contact or cooling surface is warmed to about body temperature of a human subject (i.e. about 37° C.). In some embodiments, the contact or cooling surface is warmed to a temperature above body temperature of a human subject. In some embodiments, the contact surface is kept below the pain threshold temperature (i.e. about 107° C.).

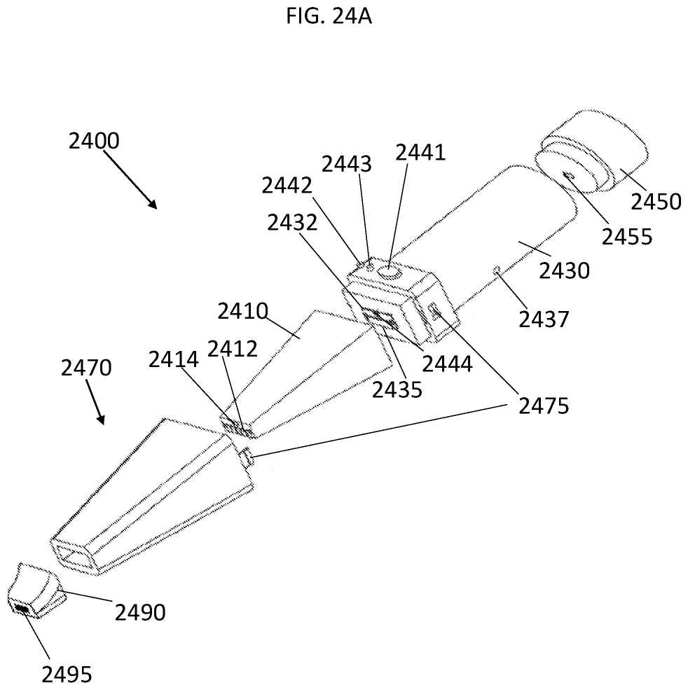

According to some embodiments, FIG. 22 depicts the temperature response skin area 2200 with a corresponding cooling curve 2210 of a cooling surface applied to the skin area. In some embodiments, the cooling surface is lowered to a first cooling temperature which is lower than a target temperature of the cooling surface. This method may be used to control the cooling curve 2200 of the skin, as the cooling curve of the skin may correlate with the perceived reduction of pain. The temperature of the cooling surface may then be raised closer to the target temperature. In some embodiments, the skin is punctured (for insertion of a needle, injection of a medicant, or the like) while the skin is being cooled to reach a target temperature. In some embodiments, the cooling surface is cooled or warmed to multiple cooling temperatures (e.g. a second cooling temperature, a third cooling temperature, etc.) as part of the method for reducing perceived pain during puncturing of the skin.

In some embodiments, a first cooling temperature of cooling surface is about 0° C. to about 20° C. In some embodiments, a first cooling temperature of cooling surface is about 0° C. to about 3° C., about 0° C. to about 5° C., about 0° C. to about 7° C., about 0° C. to about 10° C., about 0° C. to about 12° C., about 0° C. to about 15° C., about 0° C. to about 17° C., about 0° C. to about 20° C., about 3° C. to about 5° C., about 3° C. to about 7° C., about 3° C. to about 10° C., about 3° C. to about 12° C., about 3° C. to about 15° C., about 3° C. to about 17° C., about 3° C. to about 20° C., about 5° C. to about 7° C., about 5° C. to about 10° C., about 5° C. to about 12° C., about 5° C. to about 15° C., about 5° C. to about 17° C., about 5° C. to about 20° C., about 7° C. to about 10° C., about 7° C. to about 12° C., about 7° C. to about 15° C., about 7° C. to about 17° C., about 7° C. to about 20° C., about 10° C. to about 12° C., about 10° C. to about 15° C., about 10° C. to about 17° C., about 10° C. to about 20° C., about 12° C. to about 15° C., about 12° C. to about 17° C., about 12° C. to about 20° C., about 15° C. to about 17° C., about 15° C. to about 20° C., or about 17° C. to about 20° C. In some embodiments, a first cooling temperature of cooling surface is about 0° C., about 3° C., about 5° C., about 7° C., about 10° C., about 12° C., about 15° C., about 17° C., or about 20° C. In some embodiments, a first cooling temperature of cooling surface is at least about 0° C., about 3° C., about 5° C., about 7° C., about 10° C., about 12° C., about 15° C., or about 17° C. In some embodiments, a first cooling temperature of cooling surface is at most about 3° C., about 5° C., about 7° C., about 10° C., about 12° C., about 15° C., about 17° C., or about 20° C.

In some embodiments, a target cooling temperature of cooling surface is about 0° C. to about 30° C. In some embodiments, a target cooling temperature of cooling surface is about 0° C. to about 3° C., about 0° C. to about 5° C., about 0° C. to about 7° C., about 0° C. to about 10° C., about 0° C. to about 12° C., about 0° C. to about 15° C., about 0° C. to about 17° C., about 0° C. to about 20° C., about 0° C. to about 23° C., about 0° C. to about 25° C., about 0° C. to about 30° C., about 3° C. to about 5° C., about 3° C. to about 7° C., about 3° C. to about 10° C., about 3° C. to about 12° C., about 3° C. to about 15° C., about 3° C. to about 17° C., about 3° C. to about 20° C., about 3° C. to about 23° C., about 3° C. to about 25° C., about 3° C. to about 30° C., about 5° C. to about 7° C., about 5° C. to about 10° C., about 5° C. to about 12° C., about 5° C. to about 15° C., about 5° C. to about 17° C., about 5° C. to about 20° C., about 5° C. to about 23° C., about 5° C. to about 25° C., about 5° C. to about 30° C., about 7° C. to about 10° C., about 7° C. to about 12° C., about 7° C. to about 15° C., about 7° C. to about 17° C., about 7° C. to about 20° C., about 7° C. to about 23° C., about 7° C. to about 25° C., about 7° C. to about 30° C., about 10° C. to about 12° C., about 10° C. to about 15° C., about 10° C. to about 17° C., about 10° C. to about 20° C., about 10° C. to about 23° C., about 10° C. to about 25° C., about 10° C. to about 30° C., about 12° C. to about 15° C., about 12° C. to about 17° C., about 12° C. to about 20° C., about 12° C. to about 23° C., about 12° C. to about 25° C., about 12° C. to about 30° C., about 15° C. to about 17° C., about 15° C. to about 20° C., about 15° C. to about 23° C., about 15° C. to about 25° C., about 15° C. to about 30° C., about 17° C. to about 20° C., about 17° C. to about 23° C., about 17° C. to about 25° C., about 17° C. to about 30° C., about 20° C. to about 23° C., about 20° C. to about 25° C., about 20° C. to about 30° C., about 23° C. to about 25° C., about 23° C. to about 30° C., or about 25° C. to about 30° C. In some embodiments, a target cooling temperature of cooling surface is about 0° C., about 3° C., about 5° C., about 7° C., about 10° C., about 12° C., about 15° C., about 17° C., about 20° C., about 23° C., about 25° C., or about 30° C. In some embodiments, a target cooling temperature of cooling surface is at least about 0° C., about 3° C., about 5° C., about 7° C., about 10° C., about 12° C., about 15° C., about 17° C., about 20° C., about 23° C., or about 25° C. In some embodiments, a target cooling temperature of cooling surface is at most about 3° C., about 5° C., about 7° C., about 10° C., about 12° C., about 15° C., about 17° C., about 20° C., about 23° C., about 25° C., or about 30° C.

In some embodiments, the temperature of cooling surface may be monitored by at least one temperature sensor. In some embodiments, the temperature sensor may regulate a power supplied to the cooling system to adjust the temperature of the cooling surface. In some embodiments the temperature of the cooling surface is monitored and regulated to prevent the temperature of the skin from exceeding a pain threshold temperature. In some embodiments, the system ensures the skin temperature does not fall below 0° C.

In some embodiments, the temperature of the skin which is contacted by the contact or cooling surface is monitored by a temperature sensor. In some embodiments, the temperature of the skin is monitored to regulate a power supplied to the cooling system to adjust the temperature of the cooling surface. In some embodiments the temperature of the skin is monitored and regulated to prevent the contact or cooling surfaces from applying thermal values which may cause the temperature of the skin from to exceed a pain threshold temperature. In some embodiments, the system ensures the skin temperature does not fall below 10° C. Temperature sensors may include thermocouples, resistance temperature detectors, semiconductor based sensors, infrared sensors, thermistors and the like.

In some embodiments, a cooling or contact surface is provided within close proximity to the insertion site during the deployment of a needle or piercing member. In some embodiments, the contact surface completely surrounds the injection or insertion site. In some embodiments, a distance from the insertion or injection site to the contact surface represents a radius of an aperture provided through the contact surface. In some embodiments, a contact surface partially surrounds the insertion or injection site. In some embodiments, a distance from the insertion or injection site to the contact surface represents a radius of an arc length which partially surrounds the insertion or injection site.

In some embodiments, the distance from the insertion site to the contact surface is about 0.5 mm to about 4 mm. In some embodiments, the distance from the insertion site to the contact surface is about 0.5 mm to about 1 mm, about 0.5 mm to about 1.5 mm, about 0.5 mm to about 2 mm, about 0.5 mm to about 2.5 mm, about 0.5 mm to about 3 mm, about 0.5 mm to about 3.5 mm, about 0.5 mm to about 4 mm, about 1 mm to about 1.5 mm, about 1 mm to about 2 mm, about 1 mm to about 2.5 mm, about 1 mm to about 3 mm, about 1 mm to about 3.5 mm, about 1 mm to about 4 mm, about 1.5 mm to about 2 mm, about 1.5 mm to about 2.5 mm, about 1.5 mm to about 3 mm, about 1.5 mm to about 3.5 mm, about 1.5 mm to about 4 mm, about 2 mm to about 2.5 mm, about 2 mm to about 3 mm, about 2 mm to about 3.5 mm, about 2 mm to about 4 mm, about 2.5 mm to about 3 mm, about 2.5 mm to about 3.5 mm, about 2.5 mm to about 4 mm, about 3 mm to about 3.5 mm, about 3 mm to about 4 mm, or about 3.5 mm to about 4 mm. In some embodiments, the distance from the insertion site to the contact surface is about 0.5 mm, about 1 mm, about 1.5 mm, about 2 mm, about 2.5 mm, about 3 mm, about 3.5 mm, or about 4 mm. In some embodiments, the distance from the insertion site to the contact surface is at least about 0.5 mm, about 1 mm, about 1.5 mm, about 2 mm, about 2.5 mm, about 3 mm, or about 3.5 mm. In some embodiments, the distance from the insertion site to the contact surface is at most about 1 mm, about 1.5 mm, about 2 mm, about 2.5 mm, about 3 mm, about 3.5 mm, or about 4 mm.

### III. Vibrational Modes

In some embodiments, a vibrational system provides vibration to the skin. In some embodiments, the vibrations are translated through the contact or cooling surface. In some embodiments, the vibrations are applied to the skin surface as contact surface cools the skin. In some embodiments, vibration is applied to the skin surface as the temperature of the contact surface is changed. The application of vibrations may reduce the level of perceived pain as a subject receives an injection or as an object punctures the skin. The application of the vibration with the cooling of the skin may further reduce the level of perceived pain beyond what is achievable by just cooling or vibrating the skin alone.

In some embodiments, vibrations are provided at a frequency of about 20 Hz to about 40,000 Hz. In some embodiments, vibrations are provided at a frequency of about 20 Hz to about 100 Hz, about 20 Hz to about 200 Hz, about 20 Hz to about 300 Hz, about 20 Hz to about 400 Hz, about 20 Hz to about 500 Hz, about 20 Hz to about 700 Hz, about 20 Hz to about 1,000 Hz, about 20 Hz to about 10,000 Hz, about 20 Hz to about 20,000 Hz, about 20 Hz to about 40,000 Hz, about 100 Hz to about 200 Hz, about 100 Hz to about 300 Hz, about 100 Hz to about 400 Hz, about 100 Hz to about 500 Hz, about 100 Hz to about 700 Hz, about 100 Hz to about 1,000 Hz, about 100 Hz to about 10,000 Hz, about 100 Hz to about 20,000 Hz, about 100 Hz to about 40,000 Hz, about 200 Hz to about 300 Hz, about 200 Hz to about 400 Hz, about 200 Hz to about 500 Hz, about 200 Hz to about 700 Hz, about 200 Hz to about 1,000 Hz, about 200 Hz to about 10,000 Hz, about 200 Hz to about 20,000 Hz, about 200 Hz to about 40,000 Hz, about 300 Hz to about 400 Hz, about 300 Hz to about 500 Hz, about 300 Hz to about 700 Hz, about 300 Hz to about 1,000 Hz, about 300 Hz to about 10,000 Hz, about 300 Hz to about 20,000 Hz, about 300 Hz to about 40,000 Hz, about 400 Hz to about 500 Hz, about 400 Hz to about 700 Hz, about 400 Hz to about 1,000 Hz, about 400 Hz to about 10,000 Hz, about 400 Hz to about 20,000 Hz, about 400 Hz to about 40,000 Hz, about 500 Hz to about 700 Hz, about 500 Hz to about 1,000 Hz, about 500 Hz to about 10,000 Hz, about 500 Hz to about 20,000 Hz, about 500 Hz to about 40,000 Hz, about 700 Hz to about 1,000 Hz, about 700 Hz to about 10,000 Hz, about 700 Hz to about 20,000 Hz, about 700 Hz to about 40,000 Hz, about 1,000 Hz to about 10,000 Hz, about 1,000 Hz to about 20,000 Hz, about 1,000 Hz to about 40,000 Hz, about 10,000 Hz to about 20,000 Hz, about 10,000 Hz to about 40,000 Hz, or about 20,000 Hz to about 40,000 Hz. In some embodiments, vibrations are provided at a frequency of about 20 Hz, about 100 Hz, about 200 Hz, about 300 Hz, about 400 Hz, about 500 Hz, about 700 Hz, about 1,000 Hz, about 10,000 Hz, about 20,000 Hz, or about 40,000 Hz. In some embodiments, vibrations are provided at a frequency of at least about 20 Hz, about 100 Hz, about 200 Hz, about 300 Hz, about 400 Hz, about 500 Hz, about 700 Hz, about 1,000 Hz, about 10,000 Hz, or about 20,000 Hz. In some embodiments, vibrations are provided at a frequency of at most about 100 Hz, about 200 Hz, about 300 Hz, about 400 Hz, about 500 Hz, about 700 Hz, about 1,000 Hz, about 10,000 Hz, about 20,000 Hz, or about 40,000 Hz.

In some embodiments, the vibration applied to the skin surface has an amplitude ranging from about 0.3 G to 125 G, wherein G represents one gravitation unit or about 9.8 m/s² or about 294 Newtons (N). In some embodiments, the vibration applied to the skin surface has an amplitude of about 0.1 G to about 200 G. In some embodiments, the vibration applied to the skin surface has an amplitude of about 0.1 G to about 0.5 G, about 0.1 G to about 1 G, about 0.1 G to about 3 G, about 0.1 G to about 10 G, about 0.1 G to about 50 G, about 0.1 G to about 100 G, about 0.1 G to about 125 G, about 0.1 G to about 200 G, about 0.5 G to about 1 G, about 0.5 G to about 3 G, about 0.5 G to about 10 G, about 0.5 G to about 50 G, about 0.5 G to about 100 G, about 0.5 G to about 125 G, about 0.5 G to about 200 G, about 1 G to about 3 G, about 1 G to about 10 G, about 1 G to about 50 G, about 1 G to about 100 G, about 1 G to about 125 G, about 1 G to about 200 G, about 3 G to about 10 G, about 3 G to about 50 G, about 3 G to about 100 G, about 3 G to about 125 G, about 3 G to about 200 G, about 10 G to about 50 G, about 10 G to about 100 G, about 10 G to about 125 G, about 10 G to about 200 G, about 50 G to about 100 G, about 50 G to about 125 G, about 50 G to about 200 G, about 100 G to about 125 G, about 100 G to about 200 G, or about 125 G to about 200 G. In some embodiments, the vibration applied to the skin surface has an amplitude of about 0.1 G, about 0.5 G, about 1 G, about 3 G, about 10 G, about 50 G, about 100 G, about 125 G, or about 200 G. In some embodiments, the vibration applied to the skin surface has an amplitude of at least about 0.1 G, about 0.5 G, about 1 G, about 3 G, about 10 G, about 50 G, about 100 G, or about 125 G. In some embodiments, the vibration applied to the skin surface has an amplitude of at most about 0.5 G, about 1 G, about 3 G, about 10 G, about 50 G, about 100 G, about 125 G, or about 200 G.

In some embodiments, the vibration mode is perpendicular to the skin surface. In some embodiments, the vibration mode is applied in a circular motion. In some embodiments, the vibration mode is applied along a single axis of motion. In some embodiments, the vibration mode is applied parallel to the surface of the skin. In some embodiments, the vibrational mode is an eccentric rotating mode.

In some embodiments, one or more sensors measure the vibrations experienced on the skin to ensure that vibrations are being properly translated. In some embodiments, the one or more sensors measure the amplitude of the vibrations. In some embodiments, the sensors measure the frequency of the vibrations. The sensors may pressure sensors, such as piezoelectric pressure sensors. In some embodiments, the sensors are inertial sensors, such as accelerometers.

Sound produced by the vibration system may produce psychological effect to further reduce perceived pain during puncturing of the skin. In some embodiments, an audible signal is produced by the vibrational system as it vibrates a contact surface. In some embodiments, additional components are implemented to amplify the audible signal produced by the vibration system. In some embodiments, additional components produce separate audio signal. In some embodiments, the additional components produce a sound to complement the audible signal produced by the vibration system. In some embodiments the additional components produce an audio signal which comprises one or more harmonics of the vibration frequency. In some embodiments, the additional components comprise a speaker.

### IV. Wand Configurations

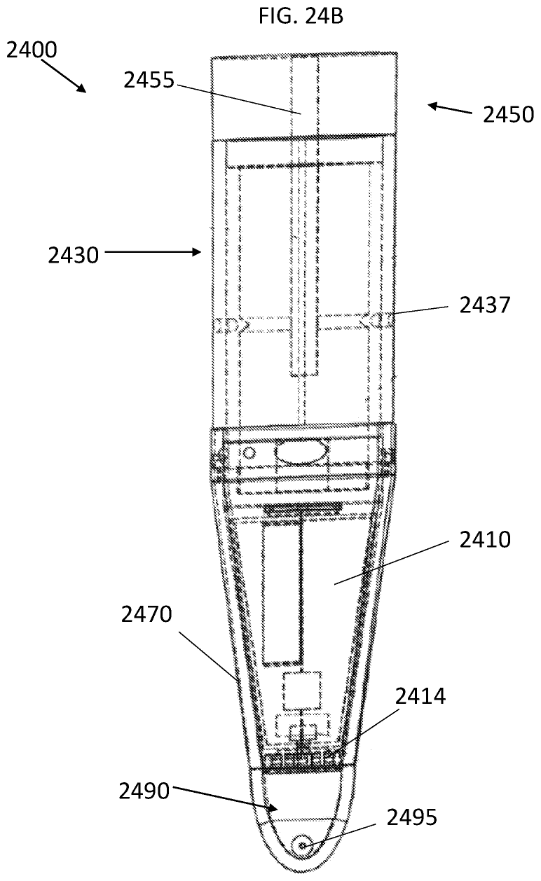

In some embodiments, with reference to FIGS. 23A-23B, a pain reduction device 2300 is provided as a wand having a handle 2350 and an applicator 2310. In some embodiments, the handle 2350 housing the internal components of the apparatus. The handle may house a cooling system, as described herein. The handle may further house a vibration system, as described herein. The handle may be configured to provide an ergonomic grip for a user.

The applicator 2310 is provided at an end of the device. The applicator may comprise one or more contact or cooling surfaces as described herein. The applicator may provide vibration to the skin of the subject. In some embodiments, the applicator comprises an aperture 2315. The aperture 2315 may allow a needle or other device to be inserted into the skin to pass through the applicator 2310. In some embodiments, the aperture is substantially circular such that the applicator completely surrounds an insertion site when applied to the skin (as depicted in FIG. 23A). In such embodiments, the contact or cooling surfaces may also completely surround the insertion site when applied to the skin. In some embodiments, the aperture partially surrounds an insertion site when applied to the skin (as depicted in FIG. 23B). In some embodiments, the region around the aperture 2315 is contoured to help guide the needle or object to be inserted into the skin.

In some embodiments, the system comprises one or more buttons 2330 to control the device. In some embodiments, a first button is configured to activate the cooling system of the device. In some embodiments, a second button is configured to activate the vibrational system of the device. In some embodiments, a first button is configured to activate the cooling and/or vibrational system of the device. In some embodiments, a second button is configured to deactivate the cooling and/or vibrational system of the device.

In some embodiments, a cooling or contact surface is provided within close proximity to the insertion site during the deployment of a needle or piercing member. In some embodiments, the aperture is substantially circular such that the applicator completely surrounds an insertion site when applied to the skin (as depicted in FIG. 23A). In some embodiments, a distance from the insertion or injection site to the contact surface represents a radius of an aperture provided through the contact surface. In some embodiments, the aperture partially surrounds an insertion site when applied to the skin (as depicted in FIG. 23B). In some embodiments, a distance from the insertion or injection site to the contact surface represents a radius of an arc length which partially surrounds the insertion or injection site.

In some embodiments, the distance from the insertion site to the contact surface is about 0.5 mm to about 4 mm. In some embodiments, the distance from the insertion site to the contact surface is about 0.5 mm to about 1 mm, about 0.5 mm to about 1.5 mm, about 0.5 mm to about 2 mm, about 0.5 mm to about 2.5 mm, about 0.5 mm to about 3 mm, about 0.5 mm to about 3.5 mm, about 0.5 mm to about 4 mm, about 1 mm to about 1.5 mm, about 1 mm to about 2 mm, about 1 mm to about 2.5 mm, about 1 mm to about 3 mm, about 1 mm to about 3.5 mm, about 1 mm to about 4 mm, about 1.5 mm to about 2 mm, about 1.5 mm to about 2.5 mm, about 1.5 mm to about 3 mm, about 1.5 mm to about 3.5 mm, about 1.5 mm to about 4 mm, about 2 mm to about 2.5 mm, about 2 mm to about 3 mm, about 2 mm to about 3.5 mm, about 2 mm to about 4 mm, about 2.5 mm to about 3 mm, about 2.5 mm to about 3.5 mm, about 2.5 mm to about 4 mm, about 3 mm to about 3.5 mm, about 3 mm to about 4 mm, or about 3.5 mm to about 4 mm. In some embodiments, the distance from the insertion site to the contact surface is about 0.5 mm, about 1 mm, about 1.5 mm, about 2 mm, about 2.5 mm, about 3 mm, about 3.5 mm, or about 4 mm. In some embodiments, the distance from the insertion site to the contact surface is at least about 0.5 mm, about 1 mm, about 1.5 mm, about 2 mm, about 2.5 mm, about 3 mm, or about 3.5 mm. In some embodiments, the distance from the insertion site to the contact surface is at most about 1 mm, about 1.5 mm, about 2 mm, about 2.5 mm, about 3 mm, about 3.5 mm, or about 4 mm.

### V. Modular Device

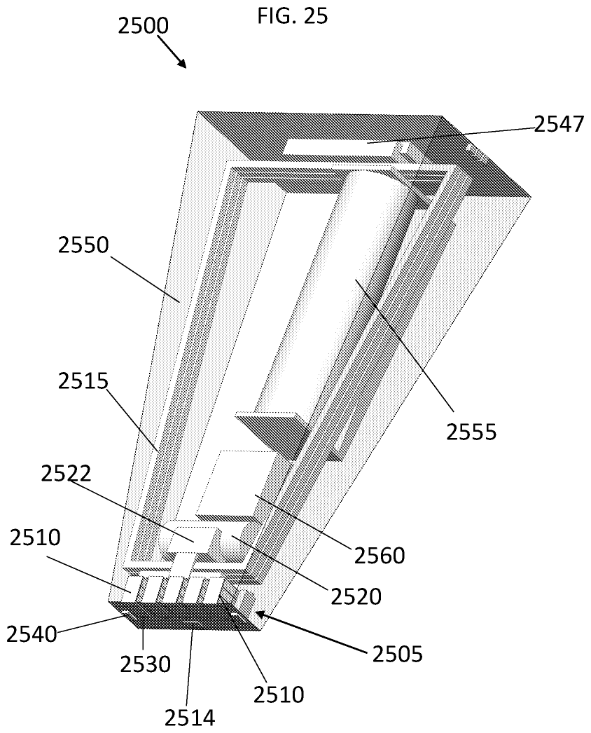

FIGS. 24A and 24B depict a modular system 2400 for reducing pain at an insertion site, according to some embodiments. In some embodiments, the system comprises a thermoelectric cooling unit 2410, a handle 2430, an end cap 2450, a protective sleeve 2470, and an applicator 2490. In some embodiments, the cooling unit comprises a battery and thermoelectric coolers 2412. In some embodiments, a protective sleeve 2470 encloses the thermoelectric cooling unit 2410, such that the unit 2410 remains sterile after use during a procedure. In some embodiments, the protective sleeve 2470 connects to the handle 2430 via a protrusion and recess system 2475 to enclose the thermoelectric cooling unit 2410.

In some embodiments, an applicator 2490 is provided at a distal end of the system and comprises a contact or cooling surface 2495. In some embodiments, the contact surface 2495 comprises an aperture to receive a puncturing device (e.g. a needle), such that the puncturing device can be placed through the contact surface and into the skin. The contact or cooling surface 2495 may be in thermal communication with the thermoelectric coolers 2412 of the cooling unit 2412. The contact surface 2495 may provide cooling and vibration to an area of the skin of a subject being treated. In some embodiments, the thermoelectric cooling unit comprises an electric communication link 2414. In some embodiments, the applicator 2490 is configured to treat a specific area of a subject. The applicator 2490 may comprise a corresponding chip to provide information to a microcontroller within the cooling unit 2410 via the communication link 2414 to inform the microcontroller as to what operations are to be performed based on the configuration of the cap 2490. In some embodiments, link 2414 receives information regarding cooling characteristics (e.g. cooling temps, times, etc.) to be provided by the cooler 2410 to the cap. In some embodiments, link 2414 receives information regarding vibration characteristics (e.g. vibration frequencies, times, amplitude, etc.) to be provided by the cooling unit 2410 to the cap. The cooling characteristics and the vibration characteristics may be considered pain reduction parameters. The chip of the applicator may provide such parameters to the microprocessor of the cooling unit.

In some embodiments, the system further comprises a handle 2430. In some embodiments, the handle comprises power contacts 2432 to receive power from a battery disposed within the cooling unit 2410. In some embodiments, the handle comprises a thermal mass. The thermal mass may be in thermal communication with a heat sink in the cooling unit 2410 via a thermal connector 2435. In some embodiments, the handle comprises one or more air vents 2437 which help dissipate heat from the thermal mass inside of the handle. In some embodiments, the thermal mass comprises through holes which are aligned with the air vents 2437.

In some embodiments, the handle comprises one or more buttons for operating the system. In some embodiments, the handle comprises a power button 2441. In some embodiments, the handle comprises a vibration and cooling activation button 2442. In some embodiments, the handle comprises a light emitting diode (LED) 2443 to indicate the status of operation of the system. In some embodiments, the handle comprises a communication contact 2444 to transmit input received from the one or more buttons to the cooling unit 2410.

In some embodiments, the system comprises an end cap 2450. The end cap may comprise a shaft 2455 for dissipating heat from the thermal mass provided in the handle. In some embodiments, the thermal mass comprises a through hole which aligns with the shaft 2455. In some embodiments, the one or more fans are provided within the shaft 2455 to further facilitate heat dissipation.

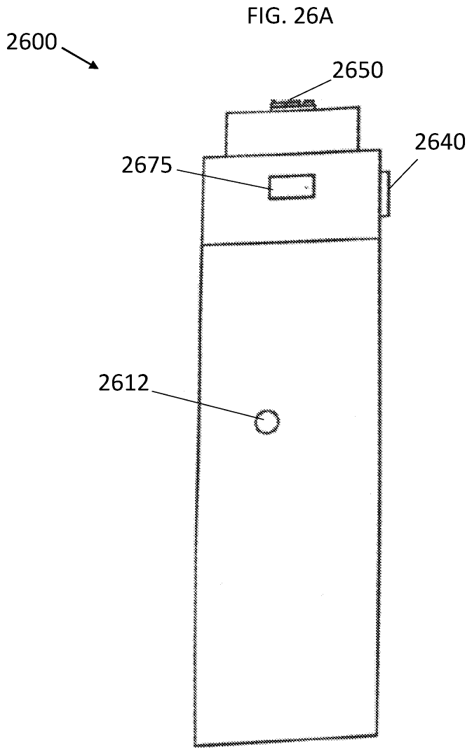

FIG. 25 depicts a thermoelectric cooling unit 2500 which of part of the modular system, according to some embodiments. In some embodiments, the cooling unit 2500 held within a protective sleeve. In some embodiments, the pain reduction device 2500 comprises one or more thermoelectric cooling units 2510. In some embodiments, the thermoelectric cooling units cool a contact surface of an applicator to cool a skin surface of a subject. In some embodiments, each cooling plate is separated by a thermal resistor. In some embodiment, the device 2500 comprises a vibrational motor 2520 to apply vibration to the skin of a subject during a procedure to be performed by the existing device. The vibration and/or cooling may be carried out by the methods as disclosed herein. In some embodiments, a vibrational mount 2522 is provided to ensure translation of vibrations from the motor 2520 to the distal end 2505 of the cooling unit.

In some embodiments, the pain reduction device 2500 comprises a distal end 2505. In some embodiments, applicator is attached to the distal end of the pain reduction device. The applicator may comprise one or more cooling surfaces provided in thermal communication with the cooling plates 2510 of the device 2500. The applicator may contact a skin surface of a patient. In some embodiments, a communication link 2514 is provided to receive information from the applicator. In some embodiments, the communication link 2514 transmits information regarding the configuration of the applicator to the microprocessor 2560 of the cooling unit 2500. The information receive may then generate a set of instruction to follow a specific procedure of transmitting cooling and/or vibration to the applicator once the cooling unit is activated.

The applicator may also be in electrical communication with electrical contacts 2540 disposed on the distal end of the cooling unit 2500 to receive power from the cooling unit. In some embodiments, the cooling tip is removably attached to the distal end 2505 of the cooling unit 2500. In some embodiments, the cooling tip is removably attached via magnetic attraction, snap fit, connection via one or more protrusions fitting into corresponding recesses, or other means of attachment. The cooling tip may be disposed of after use on the skin of the patient. This may enable the cooling unit 2500 to remain sterile.

In some embodiments, the cooling unit comprises one or more sensors 2530. The sensors may be in contact with the one or more cooling plates. In some embodiments, the sensors comprise one or more temperature sensors as described herein. In some embodiments, the sensors comprise one or more pressure sensors as described herein.

In some embodiments, the pain reduction device comprises a heat sink 2515 to dissipate heat from generated by the cooling system. In some embodiments, the heat sink 2515 is provided along an interior surface of one or more walls which form the exterior housing 2550 of the device.

In some embodiments, a proximal connector 2547 is provided at a proximal end 2507 of the pain cooling unit 2500. In some embodiments, the proximal connector 2547 provides a means of electrical communication to power source to recharge battery 2545. In some embodiments, the proximal connector 2547 provides electrical communication to a controller (not shown). The controller may be provided as part of the handle. In some embodiments, the proximal connector 2547 comprises a thermal connection, such that heat sink 2515 is in communication with a thermal mass provided in a handle.

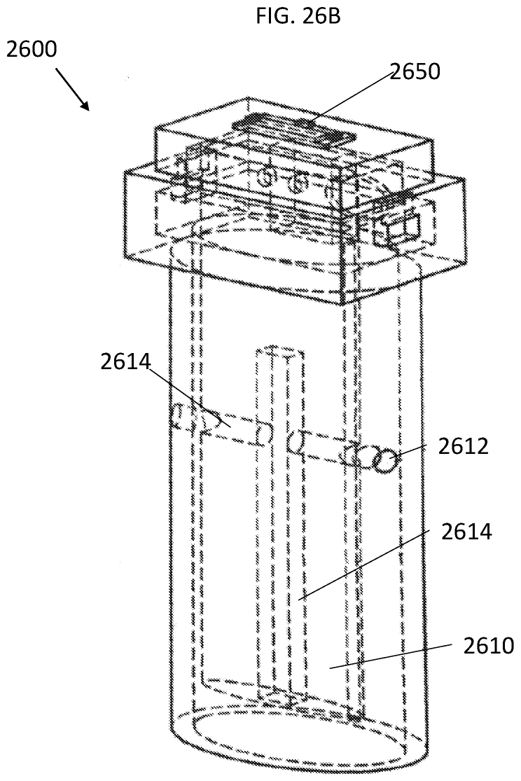

FIGS. 26A and 26B depict a handle 2600 of the modular pain reduction system, according to some embodiments. In some embodiments, the handle 2600 comprises connector 2650 to make a thermal and electrical connection to a cooling unit, as described herein. In some embodiments, the handle comprises a thermal mass 2610. When a thermoelectric cooling unit is connected via the connector 2650, heat from the cooling unit may dissipate into the thermal mass 2610. In some embodiments, thermal mass is provided with one or more air vents 2612 and one or more recesses or through-holes 2614 to facilitate heat dissipation from the thermal mass 2610.

The handle may be precooled, such that less energy is required to cool the cooling plates and cooling/contact surface of the system. The modular components may provide this unique benefit, as the handle may be separated from the cooling unit and can be comprised of parts which may be placed in a refrigeration system. Whereas cooling of a non-modular system may cause damage to the electrical components of the system. In some embodiments, the cooling system is reversible, such that a cooled thermal mass may supply power to and recharge the battery of the cooling unit.

In some embodiments, the handle 2600 is provided with one or more recesses 2675 to connect to a protective sleeve which enables enclosure and retention of the cooling device. In some embodiments, the handle comprises one or more buttons receive input from a user to activate the system. Input from the buttons 2640 may be transmitted to a microprocessor of the cooling unit via the connector 2650.

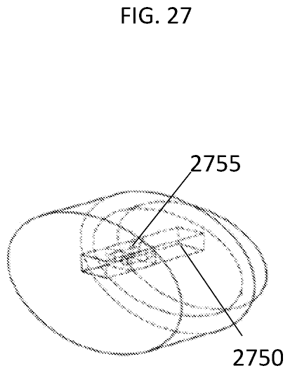

FIG. 27 depicts an end cap 2700 of the modular pain reduction system. In some embodiments, the end cap comprises a shaft 2750 which passes through the length of the cap to facilitate heat dissipation from a thermal mass provided within a handle to which the cap 2700 is attached. The shaft 2750 may be aligned with a corresponding shaft provided through the thermal mass. In some embodiments, the cap 2700 is provided with one or more fans 2755 to further facilitate heat dissipation from the thermal mass. In some embodiments, the cap comprises an electric connection with the handle, through which the fans receive power transmitted through the handle from a battery of a cooling unit. In some embodiments, the cap comprises an extruded portion 2720 which is received by a handle. In some embodiments the cap is receive by the handle via a high tolerance fit. In some embodiments, the cap is secured onto the handle by a magnetic attraction, snap fit, or other means.

### VI. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease.

As used herein, the term “about” a number refers to that number plus or minus 10% of that number. The term “about” a range refers to that range minus 10% of its lowest value and plus 10% of its greatest value.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The section headings used herein are for organizational purposes only and are not to be construed as limiting the subject matter described.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Currently Preferred Embodiment

1. In one currently preferred embodiment, the invention provides an apparatus for reducing pain during puncturing of skin of a subject, the apparatus comprising: a cooling system comprising one or more cooling surfaces to contact the skin of the subject to cause a reduction in a temperature of the skin proximal to a puncture site; and a vibration system to provide vibrations to the skin proximal to the puncture site, wherein the vibrations and the reduction of the temperature of the skin are carried out simultaneously to reduce pain at the puncture site during puncturing of the skin.

2. The apparatus of paragraph 1, wherein the reduction of temperature caused by the one or more cooling surfaces is approximately 10 to 20 degrees Celsius.

3. The apparatus of paragraph 1, wherein the reduction of temperature caused by the one or more cooling surfaces is approximately 10 degrees Celsius.

4. The apparatus of any one of paragraphs 1 to 3, wherein the one or more cooling surfaces cool the skin proximal to the puncture site to about 10 to 20 degrees Celsius.

5. The apparatus of any one of paragraphs 1 to 3, wherein the one or more cooling surfaces cool the skin proximal to the puncture site to about 15 to 20 degrees Celsius.

6. The apparatus of any one of paragraphs 1 to 3, wherein the one or more cooling surfaces cool the skin proximal to the puncture site to about 20 degrees Celsius.

7. The apparatus of any one of paragraphs 1 to 3, wherein the one or more cooling surfaces do not cool the skin proximal to the puncture site to less than 10 degrees Celsius.

8. The apparatus of any one of paragraphs 1 to 3, wherein the one or more cooling surfaces do not cool the skin proximal to the puncture site to less than 15 degrees Celsius.

9. The apparatus of any one of paragraphs 1 to 3, wherein the one or more cooling surfaces do not cool the skin proximal to the puncture site to less than 20 degrees Celsius.

10. The apparatus of any one of paragraphs 1 to 3, wherein the one or more cooling surfaces do not cool the skin proximal to the puncture site to less than 25 degrees Celsius.

11. The apparatus of paragraph 1, wherein the temperature of the skin proximal to a puncture site is reduced as the one or more cooling surface are cooled to a target temperature.

12. The apparatus of paragraph 11, wherein the target temperature is approximately 0 to 10 degrees Celsius.

13. The apparatus of paragraph 11, wherein the one or more cooling surfaces are cooled to a first temperature prior to being cooled to the target temperature. 14. The apparatus of paragraph 13, wherein the first temperature is less than the target temperature.

15. The apparatus of any one of paragraphs 11 to 13, wherein the skin is punctured as the one or more cooling surfaces being cooled to the target temperature.

16. The apparatus of any one of paragraphs 1 to 15, wherein the one or more cooling surfaces begin cooling after being contacted to the skin.

17. The apparatus of any one of paragraphs, wherein the apparatus comprises a mechanism to determine if the one or more surfaces are in contact with the skin.

18. The apparatus of paragraph 17, wherein the mechanism to determine if the one or more surfaces are in contact with the skin comprises a pressure sensor.

19. The apparatus of paragraph 18, wherein the pressure sensor is a piezoelectric pressure sensor.

20. The apparatus of any one of paragraphs 1 to 19, further comprising one or more temperature sensors to measure the temperature of the skin proximal to the puncture site.

21. The apparatus of any one of paragraphs 1 to 20, further comprising one or more temperature sensors to measure the temperature of the one or more cooling surfaces.

22. The apparatus of any one of paragraphs 1 to 21, wherein the vibrations have a frequency of approximately 100 Hertz to 1000 Hertz.

23. The apparatus of any one of paragraphs 1 to 22, wherein the vibrations have a frequency of approximately 400 Hertz to 600 Hertz.

24. The apparatus of any one of paragraphs 1 to 23, wherein the vibrations have an amplitude of 0.1 G to 100 G.

25. The apparatus of any one of paragraphs 1 to 24, wherein the apparatus comprises an aperture surrounded by the one or more cooling surfaces, wherein the aperture is placed at the puncture site and wherein an object which punctures the skin is placed through the aperture and into the puncture site.

26. The apparatus of paragraph 25, wherein the aperture comprises a radius of about 0.5 millimeters to 4 millimeters.

27. The apparatus of paragraph 25, wherein the aperture comprises a radius of about 0.5 millimeters to 1 millimeter.

28. The apparatus of any one of paragraphs 25 to 28, wherein the aperture is fully surrounded by the one or more cooling surfaces, such that the puncture site is also fully surrounded by the one or more cooling surfaces.

29. The apparatus of any one of paragraphs 25 to 28, wherein the aperture is partially surrounded by the one or more cooling surfaces, such that the puncture site is also partially surrounded by the one or more cooling surfaces.

30. The apparatus of any one of paragraphs 1 to 29, wherein the apparatus comprises a handle and an applicator provided at one end of the handle, wherein the applicator comprises the one or more cooling surfaces.

31. The apparatus of paragraph 30, wherein the applicator comprises an aperture surrounded by the one or more cooling surfaces, wherein the aperture is placed at the puncture site and wherein an object which punctures the skin is placed through the aperture and into the puncture site.

32. The apparatus of any one of paragraphs 1 to 31, further comprising a heat sink in thermal communication with the one or more cooling surfaces.

33. The apparatus of paragraph 32, wherein operation of the cooling system is reversible, such that heat stored in the heat sink is utilized to warm the one or more cooling surfaces.

34. The apparatus of paragraph 33, wherein the one or more cooling surfaces are warmed in preparation for a subsequent puncturing of the skin.

35. The apparatus of paragraph 34, wherein the subsequent puncturing of the skin occurs at a subsequent puncture site a different location from the puncture site.

36. The apparatus of paragraph 31, wherein the aperture comprises a radius of about 0.5 millimeters to 4 millimeters.

37. The apparatus of paragraph 31, wherein the aperture comprises a radius of about 0.5 millimeters to 1 millimeter.

38. The apparatus of any one of paragraphs 31 to 37, wherein the aperture is fully surrounded by the one or more cooling surfaces, such that the puncture site is also fully surrounded by the one or more cooling surfaces.

39. The apparatus of any one of paragraphs 31 to 37, wherein the aperture is partially surrounded by the one or more cooling surfaces, such that the puncture site is also partially surrounded by the one or more cooling surfaces.

40. The apparatus of paragraph 31, wherein the applicator is contoured proximal to the aperture to help guide the object which punctures the skin to the puncture site.

41. The apparatus of paragraph 40, wherein the object which punctures the skin is a needle.

42. The apparatus of paragraph 42, wherein the needle is a hypodermic needle.

43. A method of reducing pain a puncture site located on a skin of a subject, the method comprising:

applying a contact surface to the skin, proximal to the puncture site;

cooling the contact surface;

vibrating the contact surface; and

puncturing the skin at the puncture site as the contact surface is vibrating and cooling.

44. The method of paragraph 43, wherein the steps of cooling the contact surface and vibrating the contact surface are conducted after the contact surface is applied to the skin.

45. The method of paragraph 44, wherein the puncturing the skin is conducted as the contact surface is being cooled.

46. The method of any one of paragraphs 43 to 45, wherein the step of cooling the contact surface comprises cooling the contact surface to a target temperature.

47. The method of paragraph 46, wherein the target temperature is approximately 0 to 10 degrees Celsius.

48. The method of paragraph 46, wherein the cooling surface is cooled to a first temperature prior to being cooled to the target temperature.

49. The method of paragraph 48, wherein the first temperature is less than the target temperature.

50. The method of any one of paragraphs 43 to 49, further comprising a step of monitoring the temperature of the skin surface.

51. The method of paragraph 50, wherein monitoring the temperature of the skin surface is carried out by a temperature sensor.

52. The method of paragraph 50 or 51, wherein cooling of the contact surface is stopped if the temperature of the skin surface is determined to be less than 20 degrees Celsius.

53. The method of paragraph 50 or 51, wherein cooling of the contact surface is stopped if the temperature of the skin surface is determined to be less than 15 degrees Celsius.

54. The method of paragraph 50 or 51, wherein cooling of the contact surface is stopped if the temperature of the skin surface is determined to be less than 10 degrees Celsius.

55. The method of any one of paragraph 43 to 54, further comprising a step of monitoring the temperature of the contact surface.

56. The method of paragraph 55, wherein monitoring the temperature of the contact surface is carried out by a temperature sensor.

57. The method of paragraph 55 or 56, wherein cooling of the contact surface is stopped if the temperature of the skin surface is determined to be less than 10 degrees Celsius.

58. The method of paragraph 55 or 56, wherein cooling of the contact surface is stopped if the temperature of the skin surface is determined to be less than 5 degrees Celsius.

59. The method of paragraph 55 or 56, wherein cooling of the contact surface is stopped if the temperature of the skin surface is determined to be less than 0 degrees Celsius.

60. The method of any one of paragraphs 43 to 59, further comprising a step of warming the cooling surface after the step of puncturing the skin in preparation for a subsequent puncturing of the skin.

61. The method of paragraph 60, wherein the subsequent puncturing of the skin is conducted at a subsequent puncture site a location on the skin different from a location of the puncture site.

62. A pain reducing insertion apparatus, comprising:

a needle holder configured to move the needle in an axial direction with respect to a body of the insertion apparatus to insert the needle in an insertion site of an individual during deployment of the needle; and

a pain reducing system comprising a cooling system and a vibration system, wherein the cooling system is configured to cool a contact surface by conduction and the vibrator is configured to cause the contact surface to vibrate, wherein the contact surface is configured to be in a close proximity to the insertion site during the deployment of the needle.

63. The pain reducing insertion apparatus of paragraph 23, further comprising a catheter configured to couple with the needle during the deployment of the needle and wherein the catheter is inserted in the insertion site at end of the deployment.

64. The pain reducing insertion apparatus of paragraph 63, wherein the catheter is configured to be coupled to a drug delivery device.

65. The pain reducing insertion apparatus of paragraph 64, wherein the drug delivery device is an infusion set.

66. The pain reducing insertion apparatus of paragraph 23, wherein the pain reducing system does not move with respect to the body of the insertion apparatus during the deployment of the needle.

67. The pain reducing insertion apparatus of paragraph 23, wherein the cooling system is an active cooling system.

68. The pain reducing insertion apparatus of paragraph 67, wherein the active cooling system comprises a thermoelectric cooling system.

69. The pain reducing insertion apparatus of paragraph 23, wherein the cooling system is a passive cooling system.

70. The pain reducing insertion apparatus of paragraph 23, wherein the cooling system is configured to maintain the contact surface at a constant temperature.

71. The pain reducing insertion apparatus of paragraph 23, wherein the cooling system is configured to maintain the contact surface at fluctuating temperatures.

72. The pain reducing insertion apparatus of paragraph 71, wherein the fluctuating temperatures is pre-programmed.

73. The pain reducing insertion apparatus of paragraph 23, wherein the vibrating system is configured to vibrate the contact surface at a constant frequency.

74. The pain reducing insertion apparatus of paragraph 23, wherein the vibrating system is configured to vibrate the contact surface at fluctuating frequencies.

75. The pain reducing insertion apparatus of paragraph 74, wherein the fluctuating frequencies is pre-programmed.

76. A method of reducing pain during insertion of a needle at an insertion site on a skin of a patient, the method comprising:

contacting a contact surface to the skin proximal to the insertion site;

vibrating the contact surface with a vibration system;

cooling the contact surface with a cooling system; and

inserting a needle into the insertion site while the contact surface is cooling and vibrating.

77. The method of paragraph 76, wherein the needle is coupled to a catheter, and the method further comprises a step of inserting the catheter after the needle is inserted into the insertion site.

78. The method of paragraph 76 or 77, wherein the steps of cooling the contact surface and vibrating the contact surface are conducted after the contact surface is applied to the skin.

79. The method of any one of paragraphs 76 to 78, wherein the step of cooling the contact surface comprises cooling the contact surface to a target temperature.

80. The method of paragraph 79, wherein the target temperature is approximately 0 to 10 degrees Celsius.

81. The method of paragraph 79, wherein the cooling surface is cooled to a first temperature prior to being cooled to the target temperature.

82. The method of paragraph 81, wherein the first temperature is less than the target temperature.

83. The method of any one of paragraphs 76 to 82, further comprising a step of monitoring the temperature of the skin proximal to the insertion site.

84. The method of paragraph 83, wherein monitoring the temperature of the skin is carried out by a temperature sensor.

85. The method of paragraph 83 or 84, wherein cooling of the contact surface is stopped if the temperature of the skin is determined to be less than 20 degrees Celsius.

86. The method of paragraph 83 or 84, wherein cooling of the contact surface is stopped if the temperature of the skin is determined to be less than 15 degrees Celsius.

87. The method of paragraph 83 or 84, wherein cooling of the contact surface is stopped if the temperature of the skin is determined to be less than 10 degrees Celsius.

88. The method of any one of paragraphs 76 to 87, further comprising a step of monitoring the temperature of the contact surface.

89. The method of paragraph 88, wherein monitoring the temperature of the contact surface is carried out by a temperature sensor.

90. The method of paragraph 88 or 89, wherein cooling of the contact surface is stopped if the temperature of the skin surface is determined to be less than 10 degrees Celsius.

91. The method of paragraph 88 or 89, wherein cooling of the contact surface is stopped if the temperature of the skin surface is determined to be less than 5 degrees Celsius.

92. The method of paragraph 88 or 89, wherein cooling of the contact surface is stopped if the temperature of the skin surface is determined to be less than 0 degrees Celsius.

93. A system for reducing pain during puncturing of skin of a subject, the system comprising:

a cooling unit comprising:
a cooling system comprising one or more cooling plates provided at a distal end of the cooling unit, and
a vibration system comprising a vibrational motor provided at the distal end of the cooling unit; and

an applicator removably attached to the distal end of the cooling unit, the end cap comprising:
a cooling surface in thermal communication with the one or more cooling plate of the cooling unit,

wherein the cooling system of the cooling unit cools the cooling surface of the applicator.

94. The system of paragraph 93, wherein the cooling system further comprises a heat sink to dissipate heat.

95. The system of paragraph 94, further comprising a thermal mass in thermal communication with the heat sink, such that heat generated by the cooling system is dissipated into the thermal mass through the heat sink.

96. The system of paragraph 95, wherein the system further comprises a handle which is removably attached to a proximal end of the cooling unit, wherein the handle comprises the thermal mass.

97. The system of paragraph 96, wherein the cooling unit further comprises a microprocessor, and wherein the handle further comprises one or more buttons to receive an input for activating the cooling system, the vibration system, or both.

98. The system of paragraph 97, wherein the contact comprises a chip, wherein the chip transmits information to the microprocessor of the cooling unit to specify one or more pain reduction parameters.

99. The system of paragraph 98, wherein the pain reduction parameters comprise a vibration frequency, a vibration amplitude, a vibration mode, a target temperature of the cooling surface, and combinations thereof.

100. The system of any one of paragraphs 96 to 99, further comprising an end cap, wherein the end cap is removably attached to a proximal end of the handle, and wherein the end cap comprises a fan to dissipate heat from the thermal mass.

101. The system of paragraph any one of paragraphs 93 to 100, further comprising a protective sleeve to envelope the cooling unit.

102. The system of any one of paragraphs 95 to 101, wherein the system is reversible, and wherein the thermal mass is cooled prior to the attachment of the handle to the cooling unit to cool the one or more cooling plates of the cooling unit.

103. The system of paragraph 102, wherein the cooling unit further comprises a rechargeable battery, and wherein the thermal mass recharges the rechargeable battery.

1. An apparatus for reducing pain during puncturing of skin of a subject, the apparatus comprising:
a cooling system comprising one or more cooling surfaces to contact the skin of the subject to cause a reduction in a temperature of the skin proximal to a puncture site; and
a vibration system to provide vibrations to the skin proximal to the puncture site, wherein the vibrations and the reduction of the temperature of the skin are carried out simultaneously to reduce pain at the puncture site during puncturing of the skin.

2. The apparatus of claim 1, wherein the reduction of temperature caused by the one or more cooling surfaces is approximately 10 to 20 degrees Celsius.

3. The apparatus of claim 1, wherein the reduction of temperature caused by the one or more cooling surfaces is approximately 10 degrees Celsius.

4. The apparatus of claim 1, wherein the one or more cooling surfaces cool the skin proximal to the puncture site to about 10 to 20 degrees Celsius.

5. The apparatus of claim 1, wherein the one or more cooling surfaces cool the skin proximal to the puncture site to about 15 to 20 degrees Celsius.

6. The apparatus of claim 1, wherein the one or more cooling surfaces cool the skin proximal to the puncture site to about 20 degrees Celsius.

7. The apparatus of claim 1, wherein the one or more cooling surfaces do not cool the skin proximal to the puncture site to less than 10 degrees Celsius.

8. The apparatus of claim 1, wherein the one or more cooling surfaces do not cool the skin proximal to the puncture site to less than 15 degrees Celsius.

9. The apparatus of claim 1, wherein the one or more cooling surfaces do not cool the skin proximal to the puncture site to less than 20 degrees Celsius.

10. The apparatus of claim 1, wherein the one or more cooling surfaces do not cool the skin proximal to the puncture site to less than 25 degrees Celsius.

11. The apparatus of claim 1, wherein the temperature of the skin proximal to a puncture site is reduced as the one or more cooling surface are cooled to a target temperature.

12. The apparatus of claim 11, wherein the target temperature is approximately 0 to 10 degrees Celsius.

13. The apparatus of claim 11, wherein the one or more cooling surfaces are cooled to a first temperature prior to being cooled to the target temperature.

14. The apparatus of claim 13, wherein the first temperature is less than the target temperature.

15. The apparatus of any one of claims 11 to 13, wherein the skin is punctured as the one or more cooling surfaces being cooled to the target temperature.

16. The apparatus of claim 15, wherein the one or more cooling surfaces begin cooling after being contacted to the skin.

17. The apparatus of claim 16, wherein the apparatus comprises a mechanism to determine if the one or more surfaces are in contact with the skin.

18. The apparatus of claim 17, wherein the mechanism to determine if the one or more surfaces are in contact with the skin comprises a pressure sensor.

19. The apparatus of claim 18, wherein the pressure sensor is a piezoelectric pressure sensor.

20. The apparatus of claim 1, further comprising one or more temperature sensors to measure the temperature of the skin proximal to the puncture site.

21. The apparatus of claim 1 or 20, further comprising one or more temperature sensors to measure the temperature of the one or more cooling surfaces.

22. The apparatus of claim 1, wherein the vibrations have a frequency of approximately 100 Hertz to 1000 Hertz.

23. The apparatus of claim 1, wherein the vibrations have a frequency of approximately 400 Hertz to 600 Hertz.

24. The apparatus of any one of claim 1, 22, or 23, wherein the vibrations have an amplitude of 0.1 G to 100 G.

25. The apparatus of claim 1, wherein the apparatus comprises an aperture surrounded by the one or more cooling surfaces, wherein the aperture is placed at the puncture site and wherein an object which punctures the skin is placed through the aperture and into the puncture site.

26. The apparatus of claim 25, wherein the aperture comprises a radius of about 0.5 millimeters to 4 millimeters.

27. The apparatus of claim 25, wherein the aperture comprises a radius of about 0.5 millimeters to 1 millimeter.

28. The apparatus of any one of claims 25 to 27, wherein the aperture is fully surrounded by the one or more cooling surfaces, such that the puncture site is also fully surrounded by the one or more cooling surfaces.

29. The apparatus of any one of claims 25 to 27, wherein the aperture is partially surrounded by the one or more cooling surfaces, such that the puncture site is also partially surrounded by the one or more cooling surfaces.

30. The apparatus of claim 1, wherein the apparatus comprises a handle and an applicator provided at one end of the handle, wherein the applicator comprises the one or more cooling surfaces.

31. The apparatus of claim 30, wherein the applicator comprises an aperture surrounded by the one or more cooling surfaces, wherein the aperture is placed at the puncture site and wherein an object which punctures the skin is placed through the aperture and into the puncture site.

32. The apparatus of claim 1, further comprising a heat sink in thermal communication with the one or more cooling surfaces.

33. The apparatus of claim 32, wherein operation of the cooling system is reversible, such that heat stored in the heat sink is utilized to warm the one or more cooling surfaces.

34. The apparatus of claim 33, wherein the one or more cooling surfaces are warmed in preparation for a subsequent puncturing of the skin.

35. The apparatus of claim 34, wherein the subsequent puncturing of the skin occurs at a subsequent puncture site a different location from the puncture site.

36. The apparatus of claim 31, wherein the aperture comprises a radius of about 0.5 millimeters to 4 millimeters.

37. The apparatus of claim 31, wherein the aperture comprises a radius of about 0.5 millimeters to 1 millimeter.

38. The apparatus of any one of claims 31 to 37, wherein the aperture is fully surrounded by the one or more cooling surfaces, such that the puncture site is also fully surrounded by the one or more cooling surfaces.

39. The apparatus of any one of claims 31 to 37, wherein the aperture is partially surrounded by the one or more cooling surfaces, such that the puncture site is also partially surrounded by the one or more cooling surfaces.

40. The apparatus of claim 31, wherein the applicator is contoured proximal to the aperture to help guide the object which punctures the skin to the puncture site.

41. The apparatus of claim 40, wherein the object which punctures the skin is a needle.

42. The apparatus of claim 41, wherein the needle is a hypodermic needle.

43. A method of reducing pain a puncture site located on a skin of a subject, the method comprising:
applying a contact surface to the skin, proximal to the puncture site;
cooling the contact surface;
vibrating the contact surface; and
puncturing the skin at the puncture site as the contact surface is vibrating and cooling.

44. The method of claim 43, wherein the steps of cooling the contact surface and vibrating the contact surface are conducted after the contact surface is applied to the skin.

45. The method of claim 44, wherein the puncturing the skin is conducted as the contact surface is being cooled.

46. The method of claim 45, wherein the step of cooling the contact surface comprises cooling the contact surface to a target temperature.

47. The method of claim 46, wherein the target temperature is approximately 0 to 10 degrees Celsius.

48. The method of claim 46, wherein the cooling surface is cooled to a first temperature prior to being cooled to the target temperature.

49. The method of claim 48, wherein the first temperature is less than the target temperature.

50. The method of claim 43, further comprising a step of monitoring the temperature of the skin surface.

51. The method of claim 50, wherein monitoring the temperature of the skin surface is carried out by a temperature sensor.

52. The method of claim 50 or 51, wherein cooling of the contact surface is stopped if the temperature of the skin surface is determined to be less than 20 degrees Celsius.

53. The method of claim 50 or 51, wherein cooling of the contact surface is stopped if the temperature of the skin surface is determined to be less than 15 degrees Celsius.

54. The method of claim 50 or 51, wherein cooling of the contact surface is stopped if the temperature of the skin surface is determined to be less than 10 degrees Celsius.

55. The method of claim 43, further comprising a step of monitoring the temperature of the contact surface.

56. The method of claim 55, wherein monitoring the temperature of the contact surface is carried out by a temperature sensor.

57. The method of claim 55 or 56, wherein cooling of the contact surface is stopped if the temperature of the skin surface is determined to be less than 10 degrees Celsius.

58. The method of claim 55 or 56, wherein cooling of the contact surface is stopped if the temperature of the skin surface is determined to be less than 5 degrees Celsius.

59. The method of claim 55 or 56, wherein cooling of the contact surface is stopped if the temperature of the skin surface is determined to be less than 0 degrees Celsius.

60. The method of claim 43, further comprising a step of warming the cooling surface after the step of puncturing the skin in preparation for a subsequent puncturing of the skin.

61. The method of claim 60, wherein the subsequent puncturing of the skin is conducted at a subsequent puncture site a location on the skin different from a location of the puncture site.

62. A pain reducing insertion apparatus, comprising:
a needle holder configured to move the needle in an axial direction with respect to a body of the insertion apparatus to insert the needle in an insertion site of an individual during deployment of the needle; and
a pain reducing system comprising a cooling system and a vibration system, wherein the cooling system is configured to cool a contact surface by conduction and the vibrator is configured to cause the contact surface to vibrate, wherein the contact surface is configured to be in a close proximity to the insertion site during the deployment of the needle.

63. The pain reducing insertion apparatus of claim 62, further comprising a catheter configured to couple with the needle during the deployment of the needle and wherein the catheter is inserted in the insertion site at end of the deployment.

64. The pain reducing insertion apparatus of claim 63, wherein the catheter is configured to be coupled to a drug delivery device.

65. The pain reducing insertion apparatus of claim 64, wherein the drug delivery device is an infusion set.

66. The pain reducing insertion apparatus of claim 62, wherein the pain reducing system does not move with respect to the body of the insertion apparatus during the deployment of the needle.

67. The pain reducing insertion apparatus of claim 62, wherein the cooling system is an active cooling system.

68. The pain reducing insertion apparatus of claim 67, wherein the active cooling system comprises a thermoelectric cooling system.

69. The pain reducing insertion apparatus of claim 62, wherein the cooling system is a passive cooling system.

70. The pain reducing insertion apparatus of claim 62, wherein the cooling system is configured to maintain the contact surface at a constant temperature.

71. The pain reducing insertion apparatus of claim 62, wherein the cooling system is configured to maintain the contact surface at fluctuating temperatures.

72. The pain reducing insertion apparatus of claim 71, wherein the fluctuating temperatures is pre-programmed.

73. The pain reducing insertion apparatus of claim 62, wherein the vibrating system is configured to vibrate the contact surface at a constant frequency.

74. The pain reducing insertion apparatus of claim 62, wherein the vibrating system is configured to vibrate the contact surface at fluctuating frequencies.

75. The pain reducing insertion apparatus of claim 74, wherein the fluctuating frequencies is pre-programmed.

76. A method of reducing pain during insertion of a needle at an insertion site on a skin of a patient, the method comprising:
contacting a contact surface to the skin proximal to the insertion site;
vibrating the contact surface with a vibration system;
cooling the contact surface with a cooling system; and
inserting a needle into the insertion site while the contact surface is cooling and vibrating.

77. The method of claim 76, wherein the needle is coupled to a catheter, and the method further comprises a step of inserting the catheter after the needle is inserted into the insertion site.

78. The method of claim 76 or 77, wherein the steps of cooling the contact surface and vibrating the contact surface are conducted after the contact surface is applied to the skin.

79. The method of claim 76, wherein the step of cooling the contact surface comprises cooling the contact surface to a target temperature.

80. The method of claim 79, wherein the target temperature is approximately 0 to 10 degrees Celsius.

81. The method of claim 79, wherein the cooling surface is cooled to a first temperature prior to being cooled to the target temperature.

82. The method of claim 81, wherein the first temperature is less than the target temperature.

83. The method of claim 76, further comprising a step of monitoring the temperature of the skin proximal to the insertion site.

84. The method of claim 83, wherein monitoring the temperature of the skin is carried out by a temperature sensor.

85. The method of claim 83 or 84, wherein cooling of the contact surface is stopped if the temperature of the skin is determined to be less than 20 degrees Celsius.

86. The method of claim 83 or 84, wherein cooling of the contact surface is stopped if the temperature of the skin is determined to be less than 15 degrees Celsius.

87. The method of claim 83 or 84, wherein cooling of the contact surface is stopped if the temperature of the skin is determined to be less than 10 degrees Celsius.

88. The method of claim 76, further comprising a step of monitoring the temperature of the contact surface.

89. The method of claim 88, wherein monitoring the temperature of the contact surface is carried out by a temperature sensor.

90. The method of claim 88 or 89, wherein cooling of the contact surface is stopped if the temperature of the skin surface is determined to be less than 10 degrees Celsius.

91. The method of claim 88 or 89, wherein cooling of the contact surface is stopped if the temperature of the skin surface is determined to be less than 5 degrees Celsius.

92. The method of claim 88 or 89, wherein cooling of the contact surface is stopped if the temperature of the skin surface is determined to be less than 0 degrees Celsius.

93. A system for reducing pain during puncturing of skin of a subject, the system comprising:
a cooling unit comprising:
a cooling system comprising one or more cooling plates provided at a distal end of the cooling unit, and
a vibration system comprising a vibrational motor provided at the distal end of the cooling unit; and
an applicator removably attached to the distal end of the cooling unit, the end cap comprising:
a cooling surface in thermal communication with the one or more cooling plate of the cooling unit,
wherein the cooling system of the cooling unit cools the cooling surface of the applicator.

94. The system of claim 93, wherein the cooling system further comprises a heat sink to dissipate heat.

95. The system of claim 94, further comprising a thermal mass in thermal communication with the heat sink, such that heat generated by the cooling system is dissipated into the thermal mass through the heat sink.

96. The system of claim 95, wherein the system further comprises a handle which is removably attached to a proximal end of the cooling unit, wherein the handle comprises the thermal mass.

97. The system of claim 96, wherein the cooling unit further comprises a microprocessor, and wherein the handle further comprises one or more buttons to receive an input for activating the cooling system, the vibration system, or both.

98. The system of claim 97, wherein the contact comprises a chip, wherein the chip transmits information to the microprocessor of the cooling unit to specify one or more pain reduction parameters.

99. The system of claim 98, wherein the pain reduction parameters comprise a vibration frequency, a vibration amplitude, a vibration mode, a target temperature of the cooling surface, and combinations thereof.

100. The system of claim 96, further comprising an end cap, wherein the end cap is removably attached to a proximal end of the handle, and wherein the end cap comprises a fan to dissipate heat from the thermal mass.

101. The system of claim any one of claims 93 to 100, further comprising a protective sleeve to envelope the cooling unit.

102. The system of claim 95, wherein the system is reversible, and wherein the thermal mass is cooled prior to the attachment of the handle to the cooling unit to cool the one or more cooling plates of the cooling unit.

103. The system of claim 102, wherein the cooling unit further comprises a rechargeable battery, and wherein the thermal mass recharges the rechargeable battery.
